SEQUENCE LISTING (31 sequences)

SEQ ID NO: 1 - RNA, 21 nt, Artificial Sequence
  siRNA
aguuucgcgagccgagaucuu

SEQ ID NO: 2 - RNA, 21 nt, Artificial Sequence
  synthetic primer
uaccagagcagcacguugauu

SEQ ID NO: 3 - RNA, 21 nt, Artificial Sequence
  synthetic primer
ugacccaggaguacucguguu

SEQ ID NO: 4 - RNA, 21 nt, Artificial Sequence
  synthetic primer
cuucccgucguuuaucuccuu

SEQ ID NO: 5 - DNA, 21 bp, Artificial Sequence
  synthetic primer
actttcgatggtagtcgccgt

SEQ ID NO: 6 - DNA, 21 bp, Artificial Sequence
  synthetic primer
ccttggatgtggtagccgttt

SEQ ID NO: 7 - DNA, 22 bp, Artificial Sequence
  synthetic primer
gcaaagtggagattgttgccat

SEQ ID NO: 8 - DNA, 22 bp, Artificial Sequence
  synthetic primer
ccttgactgtgccgttgaattt

SEQ ID NO: 9 - DNA, 23 bp, Artificial Sequence
  synthetic primer
cccaaggccaaccgcgagaagat

SEQ ID NO: 10 - DNA, 21 bp, Artificial Sequence
  synthetic primer
gtcccggccagccaggtccag

SEQ ID NO: 11 - DNA, 20 bp, Artificial Sequence
  synthetic primer
agaggtcccagaagagccat

SEQ ID NO: 12 - DNA, 20 bp, Artificial Sequence
  synthetic primer
aagcaacttctctgccaagg

SEQ ID NO: 13 - DNA, 21 bp, Artificial Sequence
  synthetic primer
atgtcaagtgccgtctcaacg

SEQ ID NO: 14 - DNA, 21 bp, Artificial Sequence
  synthetic primer
gctgcggctttaggatgaagt

SEQ ID NO: 15 - DNA, 20 bp, Artificial Sequence
  synthetic primer
tgctgtcgctggagattgac

SEQ ID NO: 16 - DNA, 23 bp, Artificial Sequence
  synthetic primer
tggagtaagaggacacttgcgaa

SEQ ID NO: 17 - DNA, 21 bp, Artificial Sequence
  synthetic primer
agtgtgccctgacctgaaaga

SEQ ID NO: 18 - DNA, 19 bp, Artificial Sequence
  synthetic primer
tccaagagtatggcgccct

SEQ ID NO: 19 - DNA, 18 bp, Artificial Sequence
  synthetic primer
ggctggcccgataaggat

SEQ ID NO: 20 - DNA, 19 bp, Artificial Sequence
  synthetic primer
gtccccagtcatcacagcg

SEQ ID NO: 21 - DNA, 20 bp, Artificial Sequence
  synthetic primer
gaatgggactttgggctttg

SEQ ID NO: 22 - DNA, 21 bp, Artificial Sequence
  synthetic primer
gaccccaggacgatttactgc

SEQ ID NO: 23 - DNA, 20 bp, Artificial Sequence
  synthetic primer
gctcgcccagagtcactttt

SEQ ID NO: 24 - DNA, 20 bp, Artificial Sequence
  synthetic primer
gcgacgatcatttccgactt

SEQ ID NO: 25 - DNA, 21 bp, Artificial Sequence
  synthetic primer
gcttttcgtggcagcagatac

SEQ ID NO: 26 - DNA, 22 bp, Artificial Sequence
  synthetic primer
cagatttactgccccgtttgtt

SEQ ID NO: 27 - DNA, 20 bp, Artificial Sequence
  synthetic primer
agagtttgatcctggctcag

SEQ ID NO: 28 - DNA, 20 bp, Artificial Sequence
  synthetic primer
ggctgctggcacgtagttag

SEQ ID NO: 29 - protein, 410 aa, Homo sapiens
MPGVCDRAPDFLSPSEDQVLRPALGSSVALNCTAWVVSGPHCSLPSVQWLKDGLPLGIGGHYSLHEYSWVKANLSEVLVSSVLGVNVTSTEVYGAFTCSIQNISFSSFTLQRAGPTSHVAAVLASLLVLLALLLAALLYVKCRLNVLLWYQDAYGEVEINDGKLYDAYVSYSDCPEDRKFVNFILKPQLERRRGYKLFLDDRDLLPRAEPSADLLVNLSRCRRLIVVLSDAFLSRAWCSHSFREGLCRLLELTRRPIFITFEGQRRDPAHPALRLLRQHRHLVTLLLWRPGSVTPSSDFWKEVQLALPRKVQYRPVEGDPQTQLQDDKDPMLILRGRVPEGRALDSEVDPDPEGDLGVRGPVFGEPSAPPHTSGVSLGESRSSEVDVSDLGSRNYSARTDFYCLVSKDDM

SEQ ID NO: 30 - protein, 410 aa, Homo sapiens; identical to SEQ ID NO: 29

SEQ ID NO: 31 - protein, 410 aa, Homo sapiens; identical to SEQ ID NO: 29

(19) United States
(12) Patent Application Publication
(10) Pub. No.: US 2020/0281977 A1
(43) Pub. Date: Sep. 10, 2020

(54) NK OR T CELLS AND USES THEREOF

(71) Applicants: HUMANITAS MIRASOLE S.P.A., Rozzano (MI) (IT); HUMANITAS UNIVERSITY, Pieve Emanuele (MI) (IT)

(72) Inventors: Alberto MANTOVANI, Rozanna (MI) (IT); Martina MOLGORA, Rozanna (MI) (IT); Cecilia GARLANDA, Rozanna (MI) (IT); Eduardo BONAVITA, Rozanna (MI) (IT)

(73) Assignees: HUMANITAS MIRASOLE S.P.A., Rozzano (MI) (IT); HUMANITAS UNIVERSITY, Pieve Emanuele (MI) (IT)

(21) Appl. No.: 16/758,136

(22) PCT Filed: Oct. 24, 2018
(86) PCT No.: PCT/EP2018/079188

§ 371 Date: Apr. 22, 2020

(30) Foreign Application Priority Data
Oct. 24, 2017 (IT) 102017000120699

(51) Int. Cl. A61K 35/17; C07K 14/715; C07K 14/57; A61P 35/00; C07K 14/705

(52) U.S. Cl. CPC A61K 35/17; C07K 14/7155; A61K 38/00; A61P 35/00; C07K 14/70575; C07K 14/57

(57) ABSTRACT

The present invention refers to a stably or transiently IL-1R8 deficient isolated human cell, being a natural killer (NK) cell or T cell and to their medical use, preferably in the treatment of tumours and infections.

### TECHNICAL FIELD OF THE INVENTION

The present invention refers to a stably or transiently IL-1R8 deficient isolated human cell, being a natural killer (NK) cell or T cell and to their medical use, preferably in the treatment of tumours and infections.

### PRIOR ART

Interleukin-1 receptor 8 (IL-1R8, also known as single immunoglobulin IL-1R-related receptor, SIGIRR, or TIR8 [NCBI Gene ID: 59307; NM_001135053.1→NP_001128525.1; NM_001135054.1→NP_001128526.1; NM_021805.2→NP_068577.2, sequences shown below:

NCBI Reference Sequence: NP_001128525.1
GenPept Identical Proteins Graphics
>NP_001128525.1 single Ig IL-1-related receptor [Homo sapiens]
(SEQ ID NO: 29)
MPGVCDRAPDFLSPSEDQVLRPALGSSVALNCTAWVVSGPHCSLPSVQWLKDGLPLGIGGHYSLHEYSWV
KANLSEVLVSSVLGVNVTSTEVYGAFTCSIQNISFSSFTLQRAGPTSHVAAVLASLLVLLALLLAALLYV
KCRLNVLLWYQDAYGEVEINDGKLYDAYVSYSDCPEDRKFVNFILKPQLERRRGYKLFLDDRDLLPRAEP
SADLLVNLSRCRRLIVVLSDAFLSRAWCSHSFREGLCRLLELTRRPIFITFEGQRRDPAHPALRLLRQHR
HLVTLLLWRPGSVTPSSDFWKEVQLALPRKVQYRPVEGDPQTQLQDDKDPMLILRGRVPEGRALDSEVDP
DPEGDLGVRGPVFGEPSAPPHTSGVSLGESRSSEVDVSDLGSRNYSARTDFYCLVSKDDM
NCBI Reference Sequence: NP_001128526.1
GenPept Identical Proteins Graphics
>NP_001128526.1 singl Ig IL-1-related receptor [Homo sapiens]
(SEQ ID NO: 30)
MPGVCDRAPDFLSPSEDQVLRPALGSSVALNCTAWVVSGPHCSLPSVQWLKDGLPLGIGGHYSLHEYSWV
KANLSEVLVSSVLGVNVTSTEVYGAFTCSIQNISFSSFTLQRAGPTSHVAAVLASLLVLLALLLAALLYV
KCRLNVLLWYQDAYGEVEINDGKLYDAYVSYSDCPEDRKFVNFILKPQLERRRGYKLFLDDRDLLPRAEP
SADLLVNLSRCRRLIVVLSDAFLSRAWCSHSFREGLCRLLELTRRPIFITFEGQRRDPAHPALRLLRQHR
HLVTLLLWRPGSVTPSSDFWKEVQLALPRKVQYRPVEGDPQTQLQDDKDPMLILRGRVPEGRALDSEVDP
DPEGDLGVRGPVFGEPSAPPHTSGVSLGESRSSEVDVSDLGSRNYSARTDFYCLVSKDDM
NCBI Reference Sequence: NP_068577.2
GenPept Identical Proteins Graphics
>NP_068577.2 single Ig IL-1-related receptor [Homo sapiens]
(SEQ ID NO: 31)
MPGVCDRAPDFLSPSEDQVLRPALGSSVALNCTAWVVSGPHCSLPSVQWLKDGLPLGIGGHYSLHEYSWV
KANLSEVLVSSVLGVNVTSTEVYGAFTCSIQNISFSSFTLQRAGPTSHVAAVLASLLVLLALLLAALLYV
KCRLNVLLWYQDAYGEVEINDGKLYDAYVSYSDCPEDRKFVNFILKPQLERRRGYKLFLDDRDLLPRAEP
SADLLVNLSRCRRLIVVLSDAFLSRAWCSHSFREGLCRLLELTRRPIFITFEGQRRDPAHPALRLLRQHR
HLVTLLLWRPGSVTPSSDFWKEVQLALPRKVQYRPVEGDPQTQLQDDKDPMLILRGRVPEGRALDSEVDP
DPEGDLGVRGPVFGEPSAPPHTSGVSLGESRSSEVDVSDLGSRNYSARTDFYCLVSKDDM

is a member of the IL-1 receptor (ILR) family with distinct structural and functional characteristics, acting as a negative regulator of ILR and Toll-like receptor (TLR) downstream signalling pathways and inflammation¹.

The IL-1 system has a central role in both innate and adaptive immune responses and it is tightly controlled at different levels by antagonists, decoy receptors, scavengers, dominant negative molecules, miRNAs and other mechanisms, acting extracellularly or intracellularly. IL-1R8/TIR8/SIGIRR is an atypical receptor acting as a novel negative regulator of inflammatory and adaptive responses mediated by ligands of the IL-1 system. IL-1R8/TIR8/SIGIRR gene is localized on human chromosome 11 and on murine chromosome 7, and the protein (410 amino acids) is constituted by a single Ig extracellular domain with several N- and O-glycosylation sites, a transmembrane domain, an intracellular conserved TIR domain and a 95 amino acid-long tail at the C-terminal.

IL-1R8/TIR8/SIGIRR is widely expressed, in particular in epithelial tissues, such as the kidney, digestive tract, liver and lung, and in lymphoid organs by lymphoid cells.

IL-1R8/TIR8/SIGIRR has been reported to inhibit NF-kB, JNK and mTOR kinase activation following stimulation of IL-1 receptor or TLR family members. It negatively modulates the signal transduction activated by the IL-1 receptor family members IL-1R1, IL-18R, ST2, and several TLRs, such as TLR1/2, TLR3, TLR4, TLR7 and TLR9. The molecular mechanisms proposed include interference of the dimerization of IL-1R1 and IL-1RAcP through the extracellular Ig domain of IL-1R8/TIR8/SIGIRR, and binding of TIR-containing adaptor molecules through the TIR domain, which are no more available for signalling.

Natural killer (NK) cells are innate lymphoid cells which mediate resistance against pathogens and contribute to the activation and orientation of adaptive immune responses²⁻⁴. NK cells mediate resistance against haematopoietic neoplasms but are generally considered to play a minor role in solid tumour carcinogenesis⁵⁻⁷.

Several lines of evidence suggest that IL-1R8 interferes with the association of TIR module-containing adaptor molecules with signalling receptor complexes of the ILR or TLR family, tuning downstream signalling, thus negatively controlling inflammatory and immune responses and T helper cell polarization and functions^{1,8}.

It has been previously shown that CD4+ T lymphocytes express IL-1R8 (Garlanda C et al, Trends Immunol (2009); Gulen et al Immunity (2010); Bulek et al J Immunol (2009); Bozza et al J Immunol (2008)). These studies reported that IL-1R8 is a negative regulator of CD4+T lymphocytes and their helper function was amplified when IL-1R8 was genetically silenced in mice. Helper activity can be exerted by different T subsets while among T lymphocyte subsets, cytotoxic activity is mostly exerted by CD8+T subsets. The molecular mechanisms regulating the cytotoxic potential of CD8+T lymphocytes differ from those involved in CD4+T lymphocytes and the functional activities of these two cell types are different, since CD4+ T cells have helper functions and CD8+ T cells cytotoxic activity. Therefore, the regulatory role of IL-1R8 in cytotoxic T cells has still to be investigated, in particular in CD8+T lymphocytes. Moreover, IL-1R8 is the co-receptor of IL-1R5/IL-18R for IL-37 and is required for the anti-inflammatory activity of this human cytokine⁹. Deregulated activation by ILR or TLR ligands in IL-1R8-deficient mice has been associated with exacerbated inflammation and immunopathology, including selected cancers, or autoimmune diseases¹⁰.

WO2005084696 refers to the use of an agent interacting with TIR8/SIGIRR for the preparation of a therapeutic composition for treating inflammation in the gastrointestinal tract and for stimulating mucosal or epithelial immunity.

WO2007034465 refers to the novel finding that IL-1 F5 (IL-1 delta) and polypeptides derived therefrom bind to the receptor SIGIRR, with this binding interaction serving to modulate the immune response by stimulating the production of the cytokine IL-4. This induces an anti-inflammatory immune response. It has been further shown that PPARgamma is a key mediator in downstream signalling from SIGIRR following activation by the IL-1 F5 ligand. Modulation of the immune response occurs following binding of SIGIRR by IL-1 F5 in neuronal tissue and according methods for the treatment of neurodegenerative diseases are described.

It is still felt the need of a method of treating tumours by using NK or T cells.

### SUMMARY OF THE INVENTION

The present inventors found out that IL-1R8 serves as a checkpoint for NK cell maturation and effector function. Its genetic blockade unleashes NK-cell-mediated resistance to hepatic carcinogenesis, haematogenous liver and lung metastasis, and cytomegalovirus infection.

### DESCRIPTION OF THE INVENTION

Inventors found that IL-1R8 acts as a checkpoint of NK cell anti-tumor and anti-viral activity. IL-1R8 genetic inactivation in NK cells has potential translational implications in NK cell-based cell therapies.

The inventors herein show that:
IL-1R8 (mRNA and protein) is expressed by human and murine NK cells and that IL-1R8 expression is upregulated during NK cell maturation;
IL-1R8-deficiency in mice is associated with increased frequency of mature NK subsets in the blood, and lymphoid organs;
IL-1R8-deficient NK cells produce increased levels of IFNγ and show increased cytotoxic activity when stimulated in vitro with appropriate cytokines including IL-18, a member of the IL-1 family acting through IL-18R and negatively regulated by IL-1R8;
in three different models of cancer (3-MCA-induced sarcoma lung metastasis, colon cancer-derived liver metastasis and DEN-induced hepatocarcinoma), IL-1R8-deficient mice were protected: inventors observed reduced primary tumor incidence or volume and aggressiveness in the case of hepatocarcinoma and reduced number and volume of metastasis in the models of lung and liver metastasis;
depletion of NK cells abolished the protection observed in IL-1R8-deficient mice.

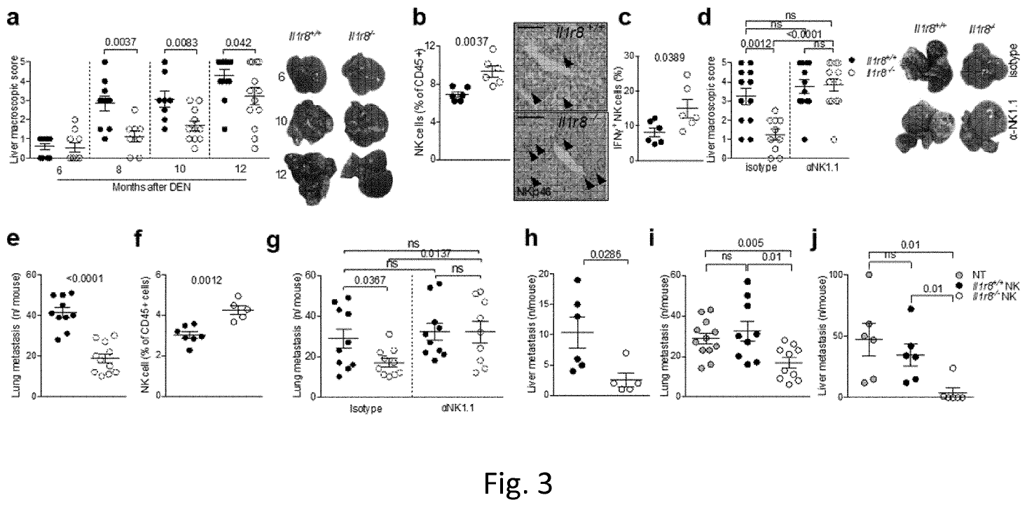

The inventors herein also show in NK cell-adoptive transfer experiments in preclinical models of liver and lung metastasis in mice that IL-1R8-deficient NK cells significantly and dramatically reduced the number and volume of metastasis (FIGS. 3i-j). This indicates that IL-1R8 deficiency is associated with increased anti-tumoral activity of NK cells.

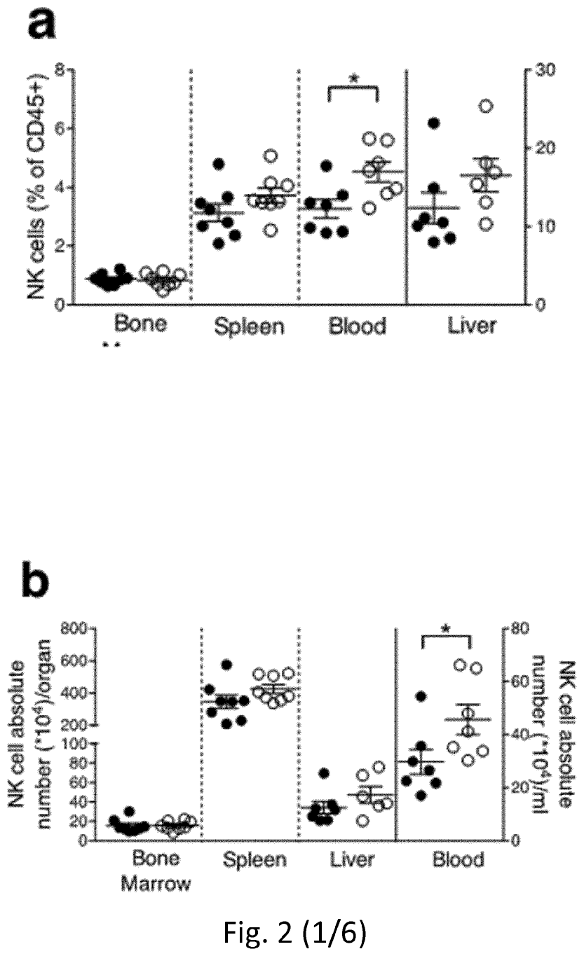

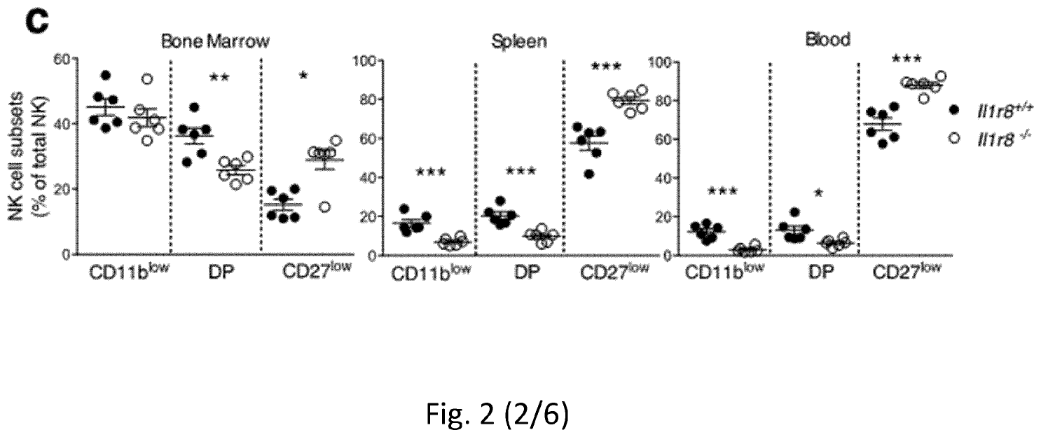

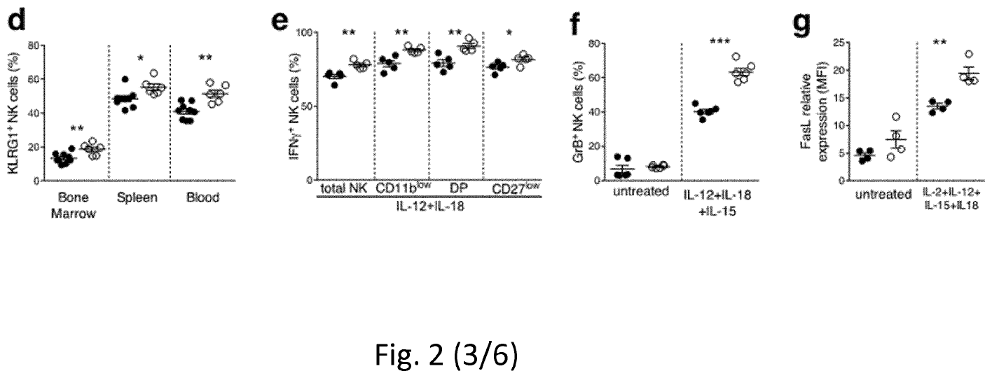

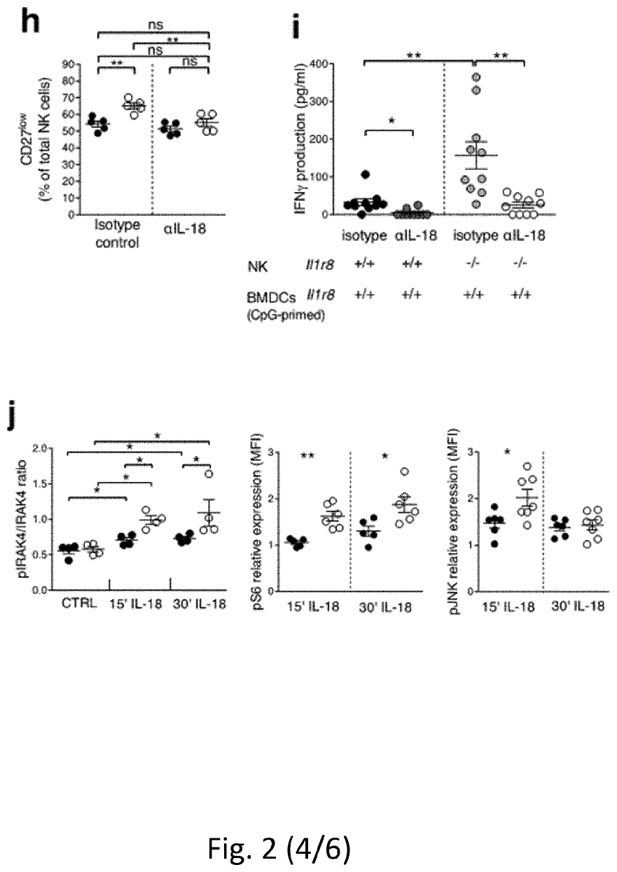

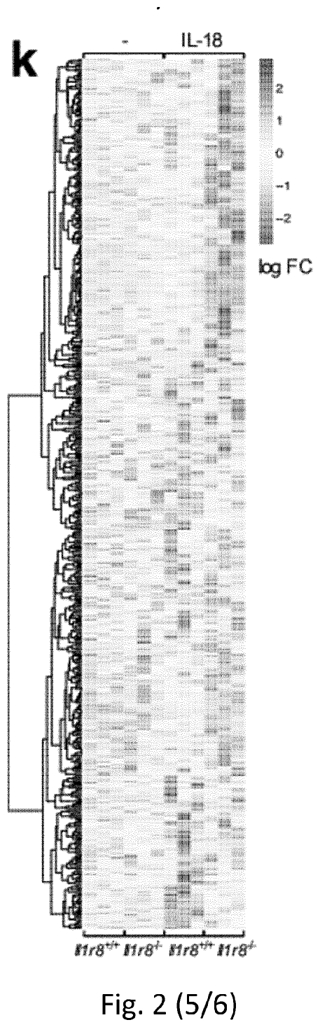

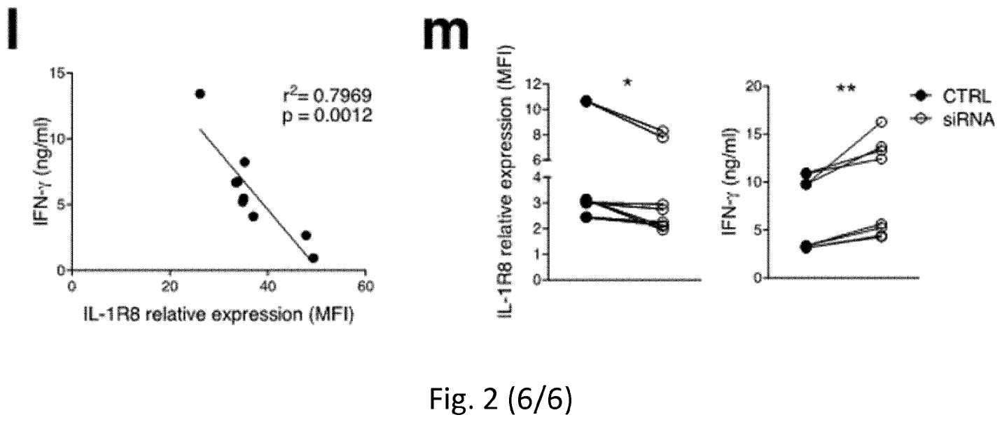

Moreover, they found that IL-1R8 expression level inversely correlates with NK cell activation in humans (FIG. 2l) and that IL-1R8 genetic inactivation through siRNA in human NK cells is associated with enhanced NK cell activation, in terms of IFNγ production (FIG. 2m) and CD69 expression, indicating that IL-1R8 serves as a negative regulator of NK cell activation and that its inactivation unleashes human NK cell effector function.

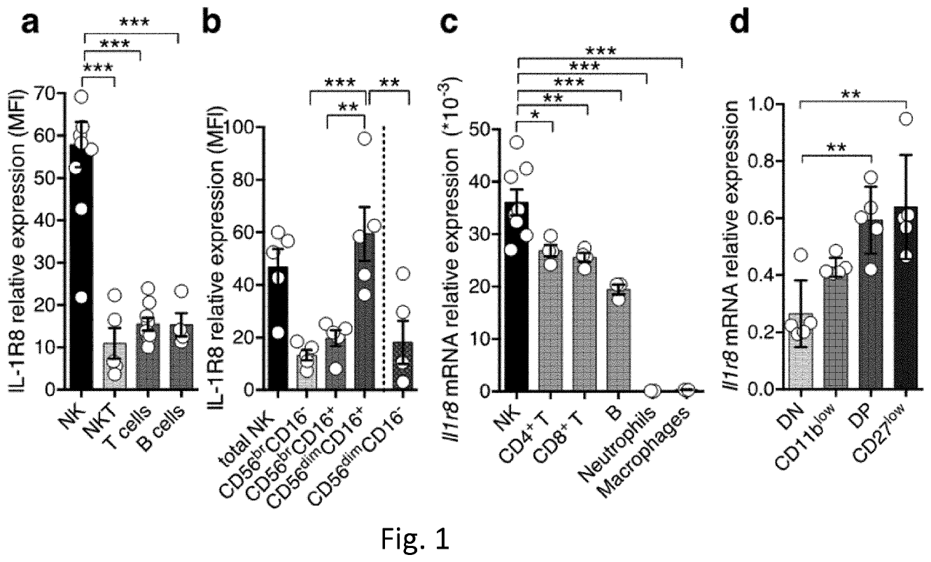

IL-1R8 is also expressed in CD8+ T cells, indicating a wider role of IL-1R8 as a checkpoint molecule and potential implication of IL-1R8-inactivation in both NK and T cells (FIG. 1a). Inventors herein also show that IL-1R8-deficiency is associated with increased CD8+ T cell proliferation, maturation and functional activation.

It is therefore an object of the invention an isolated human cell, being a natural killer (NK) cell or T cell, wherein said cell is stably or transiently deficient in the expression and/or activity of IL-1R8. Said T cell is preferably a CD8+ T cell.

Said cell preferably produces greater amounts of effector molecules involved in anti-tumour immunity, preferably interferon-gamma (IFN-γ) and/or granzyme B and/or FasL and/or express higher levels of maturation markers, preferably CD44, than cells that do express IL-1R8.

The above cell is preferably further deficient in the expression and/or activity of at least one checkpoint for NK cell maturation and/or effector function. Said at least one checkpoint for NK cell maturation and/or effector function is preferably selected from the group consisting of: CIS, KIRs, PD-1, CTLA-4, TIM-3, NKG2A, CD96, TIGIT.

Further objects of the invention are a population of cells comprising the NK cells and/or T cells as above defined and a composition comprising the cells as above defined or the population of cells as above defined, preferably further comprising at least one physiologically acceptable carrier.

The cell, or the population, or the composition as above defined are preferably for use as a medicament, more preferably for use in the treatment and/or prevention of tumour and/or metastasis, or of microbial or viral infection.

The cell or the population or the composition as above defined are preferably used in Adoptive cell transfer (ACT), cell therapy treatment, mismatched bone marrow transplantation, mismatched NK cell infusion or cytokine-induced killer (CIK) cell infusion. Said NK cell or T cell is preferably previously isolated from the same treated subject or from a different subject.

Another object of the invention is a suppressor or inhibitor of IL-1R8 expression and/or activity for medical use, preferably for use in the treatment and/or prevention of tumour and/or metastasis, or of microbial or viral infection.

Said suppressor or inhibitor is preferably at least one molecule selected from the group consisting of:

a) an antibody or a fragment thereof;

b) a polypeptide;

c) a small molecule;

d) a polynucleotide coding for said antibody or polypeptide or a functional derivative thereof;

e) a polynucleotide, such as antisense construct, antisense oligonucleotide, RNA interference construct or siRNA,

e) a vector comprising or expressing the polynucleotide as defined in d) or e);

f) a CRISPR/Cas9 component, e.g. a sgRNA

g) a host cell genetically engineered expressing said polypeptide or antibody or comprising the polynucleotide as defined in d) or e) or the component of f).

Preferably said polynucleotide is an RNA inhibitor, preferably selected from the group consisting of: siRNA, miRNA, shRNA, stRNA, snRNA, and antisense nucleic acid, more preferably the polynucleotide is at least one siRNA selected from the group consisting of: AGU UUC GCG AGC CGA GAU CUU (SEQ ID NO:1); UAC CAG AGC AGC ACG UUG AUU (SEQ ID NO:2); UGA CCC AGG AGU ACU CGU GUU (SEQ ID NO:3); CUU CCC GUC GUU UAU CUC CUU (SEQ ID NO:4) (all 5′ to 3′), or a functional derivative thereof.

Said suppressor or inhibitor is preferably used in NK and/or T cell and/or in adoptive cell transfer (ACT), cell therapy treatment, mismatched bone marrow transplantation, mismatched NK cell infusion or cytokine-induced killer (CIK) cell infusion. Preferably, said suppressor or inhibitor is preferably used for the treatment of NK and/or T cells. Said host cell is preferably an NK or T cell.

A further object of the invention is a pharmaceutical composition comprising the suppressor or inhibitor as above defined and at least one pharmaceutically acceptable carrier, and optionally further comprising a therapeutic agent.

The above tumour is preferably a solid tumor or an hematological tumor, preferably selected from the group consisting of: Colon/Rectum Cancer, Adrenal Cancer, Anal Cancer, Bile Duct Cancer, Bladder Cancer, Bone Cancer, Brain/CNS Tumors In Adults, Brain/CNS Tumors In Children, Breast Cancer, Breast Cancer In Men, Cancer of Unknown Primary, Castleman Disease, Cervical Cancer, Endometrial Cancer, Esophagus Cancer, Ewing Family Of Tumors, Eye Cancer, Gallbladder Cancer, Gastrointestinal Carcinoid Tumors, Gastrointestinal Stromal Tumor (GIST), Gestational Trophoblastic Disease, Hodgkin Disease, Kaposi Sarcoma, Kidney Cancer, Laryngeal and Hypopharyngeal Cancer, Leukemia, Acute Lymphocytic (ALL), Acute Myeloid (AML, including myeloid sarcoma and leukemia cutis), Chronic Lymphocytic (CLL), Chronic Myeloid (CML) Leukemia, Chronic Myelomonocytic (CMML), Leukemia in Children, Liver Cancer, Lung Cancer, Lung Cancer with Non-Small Cell, Lung Cancer with Small Cell, Lung Carcinoid Tumor, Lymphoma, Lymphoma of the Skin, Malignant Mesothelioma, Multiple Myeloma, Myelodysplastic Syndrome, Nasal Cavity and Paranasal Sinus Cancer, Nasopharyngeal Cancer, Neuroblastoma, Non-Hodgkin Lymphoma, Non-Hodgkin Lymphoma In Children, Oral Cavity and Oropharyngeal Cancer, Osteosarcoma, Ovarian Cancer, Pancreatic Cancer, Penile Cancer, Pituitary Tumors, Prostate Cancer, Retinoblastoma, Rhabdomyosarcoma, Salivary Gland Cancer, Sarcoma—Adult Soft Tissue Cancer, Skin Cancer, Skin Cancer—Basal and Squamous Cell, Skin Cancer—Melanoma, Skin Cancer—Merkel Cell, Small Intestine Cancer, Stomach Cancer, Testicular Cancer, Thymus Cancer, Thyroid Cancer, Uterine Sarcoma, uveal melanoma, Vaginal Cancer, Vulvar Cancer, Waldenstrom Macroglobulinemia, Wilms Tumor, more preferably the tumour is a solid tumor, preferably colorectal cancer, and the metastasis are lung or liver metastasis.

The above infection is preferably caused by one of the following viruses or bacteria: herpesviruses, preferably cytomegalovirus, Human Immunodeficiency Virus (HIV), Hepatitis C Virus (HCV), Hepatitis B Virus (HBV), West Nile virus (WNV), Salmonella, Shigella, Legionella, Mycobacterium.

Another object of the invention is a method to obtain the cell, or the population, or the composition as defined above, comprising the step of stably or transiently inhibiting or suppressing the expression and/or function of IL-1R8 in an NK or T cell or cell population comprising NK and/or T cells, and optionally further expanding in vitro the silenced population. Said T cell is preferably a CD8+ T cell. Said methods are preferably in vitro or ex vivo methods. Said NK or T cell or cell population is preferably previously purified from isolated peripheral blood mononuclear cell (PBMCs) and optionally expanded in vitro, preferably using Recombinant Human Interleukin-2 (rhIL-2).

The above method preferably further comprises the inhibition or suppression of the expression and/or function of at least one further checkpoint for NK cell maturation and/or effector function. Said at least one checkpoint for NK cell maturation and/or effector function is preferably selected from the group consisting of: CIS, KIRs, PD-1, CTLA-4, TIM-3, NKG2A, CD96, TIGIT.

In the above method the step of stably or transiently inhibiting or suppressing the expression and/or function of IL-1R8 in an NK or T cell or cell population is preferably carried out with at least one of the above defined suppressor or inhibitor.

In the context of the present invention a “CD8+ T cell” includes a cytotoxic T cell (also known as TC, cytotoxic T lymphocyte, CTL, T-killer cell, cytolytic T cell, CD8+ T-cell or killer T cell), a T lymphocyte (a type of white blood cell) that kills cancer cells, cells that are infected (particularly with viruses), or cells that are damaged in other ways. Most cytotoxic T cells express T-cell receptors (TCRs) that can recognize a specific antigen. Antigens inside a cell are bound to class I MHC molecules, which brings the antigen to the surface of the cell where they can be recognized by the T cell. In order for the TCR to bind to the class I MHC molecule, the former must be accompanied by a glycoprotein called CD8, which binds to the constant portion of the class I MHC molecule. Therefore, these T cells are defined as CD8+ T cells.

In the context of the present invention, a cell deficient in the expression and/or activity of IL-1R8 is a cell in which the levels of IL-1R8 (protein and/or mRNA) are reduced or completely inhibited permanently or transiently. A cell deficient in the expression and/or activity of IL-1R8 may be obtained e.g. by silencing using CRISPR/Cas9 system, siRNA, peptides or antibodies interfering with the interaction with other ILR/TLR receptors. Said deficient cell may be e.g. transformed using sgRNA, preferably said sgRNA being delivered into the cells with a CRISPR-Cas9 system.

In one embodiment, the NK and/or T cells deficient in the expression and/or activity of IL-1R8 express no detectable IL-1R8. In another embodiment, the NK and/or T cells deficient in the expression and/or activity of IL-1R8 express no immunologically detectable IL-1R8. In one embodiment, the NK and/or T cells deficient in the expression and/or activity of IL-1R8 express no detectable IL-1R8 mRNA. The NK and/or T cells deficient in the expression and/or activity of IL-1R8 (or lacking functional IL-1R8) can be prepared using any conventional method. In some embodiments, a cell deficient in the expression and/or activity of IL-1R8 is obtained by inhibiting or blocking IL-1R8 expression by, e.g., gene deletion, gene disruption, siRNA, shRNA or antisense approaches. In other embodiments, a cell deficient in the expression and/or activity of IL-1R8 is obtained by inhibiting or blocking IL-1R8 activity by, e.g., a IL-1R8 antagonist or antibody. In certain embodiments, a cell deficient in the expression and/or activity of IL-1R8 is obtained by blocking the expression of endogenous IL-1R8 by genetically modifying the immune cell. Although in some cases homologous recombination is used, in particular cases non-homologous end joining is used to edit the genome. Any suitable protocol to modify the genome of a particular immune cell is useful, although in specific embodiments gene modification is achieved using an engineered nuclease such as a zinc finger nuclease (ZFP), TALE-nuclease (TALEN), or CRISPR/Cas nuclease. Engineered nuclease technology is based on the engineering of naturally occurring DNA-binding proteins. For example, engineering of homing endonucleases with tailored DNA-binding specificities has been described, (see, Chames, et al. (2005) Nucleic Acids Res. 33(20):e178; Arnould, et al. (2006) J. Mol. Biol. 355:443-458). In addition, engineering of ZFPs has also been described. See, e.g., U.S. Pat. Nos. 6,534,261; 6,607,882; 6,824,978; 6,979,539; 6,933,113; 7,163,824; and 7,013,219.

All the above definitions of “cell deficient in the expression and/or activity” apply, mutatis mutandis, also to the “cells deficient in the expression and/or activity of at least one checkpoint for NK cell maturation and/or effector function”.

The term “checkpoint for NK cell maturation and/or effector function” includes molecules which are fundamental for the regulation of immune-mediated responses e.g. the molecule known as CIS (cytokine-inducible SH2-containing protein), KIRs (killer cell immunoglobulin-like receptor), PD-1, CTLA-4, TIM-3, NKG2A, CD96, TIGIT (Hsu J et al, JCI (2018) https://doi.org/10.1172/JCI99317.; Guillerey C et al, Nat Immunol (2016) https://doi.org/10.1038/ni.3518; Delconte R B et al, Nat Immunol (2016) https://doi.org/10.1038/ni.3470). PD-1 blockade is known to favour an immune reactivation, being therefore protective and curative in tumor models and oncological patients; the other molecules (i.e. CTLA-4, PD-L1, KIRs, TIM-3, NKG2A, CD96, TIGIT, CIS) regulating different pathways and acting through different mechanisms, were previously described as inhibitory molecules in NK cells. Most of them are already in use in clinics, others are under development (e.g. CIS, CD96). PD-1 is the checkpoint molecule mostly used in the clinic and for which tools are available for preclinical studies in the mouse. The role of PD-1 as a checkpoint molecule of NK cells has recently been published (Hsu J et al, JCI (2018)). PD-1 is expressed in terminally differentiated and exhausted cytotoxic lymphocytes and it is induced upon chronic activation and in the tumor microenvironment as a mechanism of immunosuppression (Freeman G J et a. JEM (2000)). PD-1-dependent immune inhibitory activity depends on the interaction with the ligand (PD-L1) expressed on the target cell, in particular tumoral cells (Freeman G J et a. JEM (2000); Hsu J et al, JCI (2018)). Therefore, the inhibition of the PD-1/PD-L1 axis with checkpoint inhibitors (anti-PD-1 or anti-PD-L1 blocking antibodies) can be addressed only in presence of the cytotoxic cell type (e.g. NK cells, CD8+ T cells) and a target (e.g. tumoral cell).

In the context of the present invention an “effector molecule involved in anti-tumour immunity” is a molecule which mediates fundamental mechanisms of the immune response against tumor cells. Preferably it can be interferon-gamma (IFN-γ), granzyme B, FasL.

The population of cells according to the invention preferably comprises at least 50% of the NK cells and/or T cells as defined above.

In one embodiment, the composition or the cell population as defined above comprises more than 50% of NK and/or T cells deficient in the expression and/or activity of IL-1R8. In another embodiment, the composition or the cell population comprises more than 70% of NK and/or T cells deficient in the expression and/or activity of IL-1R8. In another embodiment, the composition or cell population comprises more than 80% of NK and/or T cells deficient in the expression and/or activity of IL-1R8.

The T cell of the invention is preferably a CD8⁺ T cell.

The above-mentioned cytokines are observed in vivo. Therefore, the expression “said cell produces” includes not only the direct production but also the indirect production of cytokines, relating to the final effect of the tumoral process, controlled differently between the two animal groups.

Every known method for obtaining/expanding mature NK or T cells may be used. Several strategies have indeed been developed to obtain/expand mature NK cells in vitro (see e.g. Fang F. et al. Semin Immunol 31 (2017) 37-54; Davis Z. B. et al. Semin Immunol 31 (2017) 64-75). As a way of example, NK cells may be purified from PBMCs and expanded in vitro using rhIL-2. IL-1R8 may be then silenced using any silencing method, e.g. CRISPR/Cas9 system or siRNA or neutralized with mAb. Pretreatment with cytokines may be preferably considered and NK or T cells may be infused in patients by any convenient administration route, e.g. through intravenous or intra-arterial injection. (see for instance Koehl U, et al. Front Oncol. 2013 May 17; 3:118. doi: 10.3389/fonc.2013.00118. eCollection 2013. Granzim N. et al. Front Immunol. 2017 Apr. 26; 8:458. doi: 10.3389/fimmu.2017.00458. eCollection 2017).

In the context of the present invention, “IL-1R8 activity” or “activity of IL-R8” comprises e.g. the interaction with other IL-1R family members and TLR family members, the negative regulation of TLR family members activation and signal transduction, inhibition of NF-kB, JNK and/or mTOR kinas activation, negative modulation of the signal transduction activated by the IL-1 receptor family member, e.g. IL-1R1, IL-18R, ST2, and TLRs, e.g. TLR1/2, TLR3, TLR4, TLR7 and/or TLR9.

IL-1R8 is a membrane receptor that interacts with other IL-1R family members and TLR family members, negatively regulating their activation and signal transduction. IL-1R8 activity has been e.g. inhibited by the present inventors through genetic deficiency in mice and genetic silencing using siRNA in humans using Dharmacon™Accell™ siRNA technology.

In addition, IL-1R8 activity may be inhibited by silencing using CRISPR/Cas9 system, other siRNA, by peptides or antibodies interfering with the interaction with other ILR/TLR receptors, as described for instance by Fang F. et al. Semin Immunol 31 (2017) 37-54. In the context of the present invention the term “activity” and “function” are interchangeable.

The NK cells of the invention include NK progenitors and mature and functional NK cells.

The NK progenitor cells can be differentiated into mature and functional NK cells recognizing a desired target by specific receptors on their surface known to the expert in the field (e.g. NKG2D, DNAM-1, NCRs, KIR-receptors). These mature and functional NK cells can be generated in vitro by extending the culture period 2-3 more weeks. However, as cellular therapeutic the injection of the primitive progenitors and maturation in vivo is preferred. These NK cells can be used in the treatment of tumors, cancer, in particular leukemias, ovarian, colon and skin cancers, Breast, Brain and Lung cancers, Cervical cancer and metastases of all kinds of cancer, particularly to the liver, as well as all viral diseases, in particular HIV, HCV, and other chronic viral diseases.

Doses for such pharmaceutical compositions are generally expressed in the number of viable cells present in such a composition. Said number should be between 1-9×10⁶ NK-initiating cells or >1-10×10⁸ mature NK-cells or 1-9×10⁶ T cells per kg body weight of a subject to be treated. After pretreatment with cytokines, NK cells according to the invention may be infused in patients through intravenous or intra-arterial injection (see for instance Koehl U, et al. Front Oncol. 2013 May 17; 3:118. doi: 10.3389/fonc.2013.00118. eCollection 2013. Granzim N. et al. Front Immunol. 2017 Apr. 26; 8:458. doi: 10.3389/fimmu.2017.00458. eCollection 2017).

The polynucleotides as above described, as e.g. the siRNAs, may further comprise dTdT or UU 3′-overhangs, and/or nucleotide and/or polynucleotide backbone modifications as described elsewhere herein. In the context of the present invention, the term “polynucleotide” includes DNA molecules (e.g., cDNA or genomic DNA) and RNA molecules (e.g., mRNA, siRNA, shRNA) and analogs of the DNA or RNA generated using nucleotide analogs. The polynucleotide may be single-stranded or double-stranded. The RNAi inhibitors as above defined are preferably capable of hybridizing to all or part of specific target sequence. Therefore, RNAi inhibitors may be fully or partly complementary to all of or part of the target sequence. The RNAi inhibitors may hybridize to the specified target sequence under conditions of medium to high stringency. An RNAi inhibitors may be defined with reference to a specific sequence identity to the reverse complement of the sequence to which it is intended to target. The antisense sequences will typically have at least about 75%, preferably at least about 80%, at least about 85%, at least about 90%, at least about 95% or at least about 99% sequence identity with the reverse complements of their target sequences.

The term polynucleotide and polypeptide also includes derivatives and functional fragments thereof. The polynucleotide may be synthesized using oligonucleotide analogs or derivatives (e.g., inosine or phosphorothioate nucleotides).

In the context of the present invention, the genes as above defined (as IL-1R8) are preferably characterized by the sequences identified by their NCBI Gene ID and Gen Bank Accession numbers. However, they include also corresponding orthologous or homologous genes, isoforms, variants, allelic variants, functional derivatives, functional fragments thereof.

In the context of the present invention the term “gene” also includes corresponding orthologous or homologous genes, isoforms, variants, allelic variants, functional derivatives, functional fragments thereof. The expression “protein” is intended to include also the corresponding protein encoded from a corresponding orthologous or homologous genes, functional mutants, functional derivatives, functional fragments or analogues, isoforms thereof.

In the context of the present invention, the term “polypeptide” or “protein” includes:

i. the whole protein, allelic variants and orthologs thereof;

ii. any synthetic, recombinant or proteolytic functional fragment;

iii. any functional equivalent, such as, for example, synthetic or recombinant functional analogues.

The term “analogue” as used herein referring to a protein means a modified peptide wherein one or more amino acid residues of the peptide have been substituted by other amino acid residues and/or wherein one or more amino acid residues have been deleted from the peptide and/or wherein one or more amino acid residues have been deleted from the peptide and or wherein one or more amino acid residues have been added to the peptide. Such addition or deletion of amino acid residues can take place at the N-terminal of the peptide and/or at the C-terminal of the peptide.

A “derivative” may be a nucleic acid molecule, as a DNA molecule, coding the polynucleotide as above defined, or a nucleic acid molecule comprising the polynucleotide as above defined, or a polynucleotide of complementary sequence. In the context of the present invention the term “derivatives” also refers to longer or shorter polynucleotides and/or polypeptides having e.g. a percentage of identity of at least 41%, 50%, 60%, 65%, 70% or 75%, more preferably of at least 85%, as an example of at least 90%, and even more preferably of at least 95% or 100% with the sequences herein mentioned or with their complementary sequence or with their DNA or RNA corresponding sequence. The term “derivatives” and the term “polynucleotide” also include modified synthetic oligonucleotides. The modified synthetic oligonucleotide are preferably LNA (Locked Nucleic Acid), phosphoro-thiolated oligos or methylated oligos, morpholinos, 2′-O-methyl, 2′-O-methoxyethyl oligonucleotides and cholesterol-conjugated 2′-O-methyl modified oligonucleotides (antagomirs). The term “derivative” may also include nucleotide analogues, i.e. a naturally occurring ribonucleotide or deoxyribonucleotide substituted by a non-naturally occurring nucleotide. The term “derivatives” also includes nucleic acids or polypeptides that may be generated by mutating one or more nucleotide or amino acid in their sequences, equivalents or precursor sequences. The term “derivatives” also includes at least one functional fragment of the polynucleotide. In the context of the present invention “functional” is intended for example as “maintaining their activity”. The above defined antibodies comprise human and animal monoclonal antibodies or fragments thereof, single chain antibodies and fragments thereof and miniantibodies, bispecific antibodies, diabodies, triabodies, or di-, oligo- or multimers thereof. Also included are peptidomimetics or peptides derived from the antibodies according to the invention, e.g. they comprise one or several CDR regions, preferably the CDR3 region. Further included are human monoclonal antibodies and peptide sequences which, based on a structure activity connection, are produced through an artificial modeling process (Greer J. et al., J. Med. Chem., 1994, Vol. 37, pp. 1035-1054).

Preferably, the antibody is selected from the group consisting of an intact immunoglobulin (or antibody), a Fv, a scFv (single chain Fv fragment), a Fab, a F(ab′)₂, an antibody-like domain, an antibody-mimetic domain, a single antibody domain, a multimeric antibody, a peptide or a proteolytic fragment containing the epitope binding region. The term “antibody” in the present invention is used in the most general sense, and encompasses various antibodies and antibody mimetic structures, including, but not limited to, monoclonal antibodies, polyclonal antibodies, multispecific antibodies (e.g., bispecific antibodies), human antibodies, humanized antibodies, deimmunized antibodies, chimeric antibodies, nanobodies, antibody derivatives, antibody fragments, anticalines, DARPins, affibody, affilins, affimers, affitines, alphabody, avimers, fynomers, minibodies and other binding domains, provided that they show desired binding activity for the antigen. An “antibody fragment” refers to a molecule other than an intact antibody that comprises a portion of an intact antibody that binds the antigen to which the intact antibody binds. Examples of antibody fragments include, but are not limited to, Fv, Fab, Fab′, Fab′-SH, F(ab′)2; diabody; linear antibodies; single-chain antibody molecules (e.g. scFv); and multispecific antibodies consisting of antibody fragments. Fv of VH and VL are also called “nanobodies”. The term “mimetic antibody” refers to those organic compounds or binding domains that are not antibody derivatives but that can specifically bind to an antigen, in the same way of the antibodies. They include anticalines, DARPins, affibody, affilins, affimers, affitines, alphabody, avimers, fynomers, minibodies, and others. The term “chimeric” antibody refers to an antibody in which a portion of the heavy and/or light chain is derived from one specific source or species, while the remainder of the heavy and/or light chain is derived from a different source or species.

The terms “full-length antibody,” “intact antibody” and “whole antibody” are used herein interchangeably to refer to an antibody having a structure substantially similar to a native antibody structure or having heavy chains that contain a Fc region as defined herein. A “human antibody” is one that possesses an amino acid sequence which corresponds to that of an antibody produced by a human being or a human cell or derived from a non-human source that uses repertoires of human antibodies or other sequences encoding human antibodies. This definition of a human antibody specifically excludes a humanized antibody comprising non-human antigen-binding residues. In humans, the antibody isotypes are IgA, IgD, IgE, IgG and IgM. An antibody “humanized” refers to a chimeric antibody comprising amino acid residues from non-human hypervariable regions (HVR) and amino acid residues from the remaining human regions (FR: Framework Regions). In certain embodiments, a humanized antibody will comprise substantially at least an entire variable domain, and typically two, in which all or substantially all of the HVRs (for example, CDRs) correspond to those of a non-human antibody, and all or substantially all of the FRs correspond to those of a human antibody. A humanized antibody optionally may comprise at least a portion of an antibody constant region derived from a human antibody. A “humanized form” of an antibody, for example, a non-human antibody, refers to an antibody subjected to humanization. An antibody “deimmunized” is an antibody with reduced immunogenicity based on the destruction of HLA binding, a basic requirement for the stimulation of T cells. A monoclonal antibodies to be used according to the present invention can be for example produced by a variety of techniques, including, but not limited to, the hybridoma method, methods based on recombinant DNA, phage display methods, and methods that use transgenic animals containing all or part of human immunoglobulin loci. In the context of the present invention, the antibody of the present invention includes modifications of the antibody according to the present invention able to maintain the specificity mentioned above. These changes include, for example, the conjugation to effector molecules such as chemotherapeutic or cytotoxic agents, and/or detectable reporter portions.

Bispecific antibodies are macromolecular, heterobifunctional cross-linkers having two different binding specificities within one single molecule. In this group belong, e.g., bispecific (bs) IgGs, bs IgM-IgAs, bs IgA-dimers, bs (Fab′)2, bs(scFv)2, diabodies, and bs bis Fab Fc (Cao Y. and Suresh M. R., Bioconjugate Chem., 1998, Vol. 9, pp. 635-644).

By peptidomimetics, protein components of low molecular weight are understood which imitate the structure of a natural peptide component, or of templates which induce a specific structure formation in an adjacent peptide sequence (Kemp D S, Trends Biotechnol., 1990, pp. 249-255). The peptidomimetics may, e.g., be derived from the CDR3 domains. Methodical mutational analysis of a given peptide sequence, i.e. by alanine or glutamic acid scanning mutational analysis, may be used. Another possibility to improve the activity of a certain peptide sequence is the use of peptide libraries combined with high throughput screening.

The term antibodies may also comprise agents which have been obtained by analysis of data relating to structure-activity relationships. These compounds may also be used as peptidomimetics (Grassy G. et al., Nature Biotechnol., 1998, Vol. 16, pp. 748-752; Greer J. et al., J. Med. Chem., 1994, Vol. 37, pp. 1035-1054).

The term antibody may also include proteins produced by expression of an altered, immunoglobulin-encoding region in a host cell, e.g. “technically modified antibodies” such as synthetic antibodies, chimeric or humanized antibodies, or mixtures thereof, or antibody fragments which partially or completely lack the constant region, e.g. Fv, Fab, Fab′ or F(ab)′2 etc. In these technically modified antibodies, e.g., a part or parts of the light and/or heavy chain may be substituted. Such molecules may, e.g., comprise antibodies consisting of a humanized heavy chain and an unmodified light chain (or chimeric light chain), or vice versa. The terms Fv, Fc, Fd, Fab, Fab′ or F(ab)₂ are used as described in the prior art (Harlow E. and Lane D., in “Antibodies, A Laboratory Manual”, Cold Spring Harbor Laboratory, 1988).

The present invention also comprises the use of Fab fragments or F(ab)₂ fragments which are derived from monoclonal antibodies (mAb), which are directed against IL-1R8 or other checkpoint for NK cell maturation and/or effector function. Preferably, the heterologous framework regions and constant regions are selected from the human immunoglobulin classes and isotypes, such as IgG (subtypes 1 to 4), IgM, IgA and IgE. In the course of the immune response, a class switch of the immunoglobulins may occur, e.g. a switch from IgM to IgG; therein, the constant regions are exchanged, e.g. μ from to γ. A class switch may also be caused in a directed manner by means of genetic engineering methods (“directed class switch recombination”), as is known from the prior art (Esser C. and Radbruch A., Annu. Rev. Immunol., 1990, Vol. 8, pp. 717-735). However, the antibodies according to the present invention need not comprise exclusively human sequences of the immunoglobulin proteins.

The antibodies of the present invention also include those for which binding characteristics have been improved by direct mutations, affinity maturation methods, phage display. The affinity or specificity can be modified or improved by mutations in any of the antibody CDRs of the present invention. The term “variable region” or “variable domain” refers to the domain of a heavy or light chain of antibody that is involved in the binding of the antibody to the antigen. The variable domains (or regions) of the heavy and light chain (VH and VL, respectively) of a native antibody generally have similar structures, each domain comprising four framework conserved regions (FR) and three hypervariable regions (HVR, see, for example, Kindt et al. Kuby Immunology, 6th ed., W.H. Freeman and Co., page 91, 2007). A single VH or VL domain can be sufficient to confer antigen binding specificity. Moreover, it is possible to isolate antibodies that bind to a specific antigen using a VH or VL domain from an antibody that binds the antigen to screen a library of complementary VL or VH domains, respectively (see, for example, Portolano et al., J. Immunol. 150:880-887, 1993; Clarkson et al., Nature 352:624-628, 1991).

The antibody-like domain comprises binding proteins structurally related to antibodies, such as T cell receptors. The antibodies of the present invention also include functional equivalents that include polypeptides with amino acid sequences substantially identical to the amino acid sequence of the variable or hypervariable regions of the antibodies of the present invention. “The percent (%) amino acid sequence identity” with respect to a reference polypeptide sequence is defined as the percentage of amino acid residues in a candidate sequence that are identical to the amino acid residues in the reference polypeptide sequence, after aligning the sequences and introducing gaps, if necessary, to achieve maximum percent sequence identity, and not considering any conservative substitutions as part of the sequence identity. The alignment in order to determine the percent of amino acid sequence identity can be achieved in various ways that are within the skill in the art, for instance, using publicly available computer software such as BLAST, BLAST-2, ALIGN or Megalign software (DNASTAR). Those skilled in the art can determine appropriate parameters for aligning sequences, including any algorithms needed to achieve maximal alignment over the full-length of the sequences being compared. The antibody of the invention may e.g. have a dissociation constant (K_{D}) of <100 nM, <10 nM, <1 nM, <0.1 nM, <0.01 nM, or <0.001 nM or less, e.g. from 10⁻⁸ M to 10⁻⁻¹³ M, e.g., from 10⁻⁹ M to 10⁻¹³ M. Recombinant and/or biotechnological derivatives as well as fragments of the antibodies described above are included within the invention, provided that the binding activity of the antibodies and their functional specificity is maintained.

In the context of the present invention, the “cancer” or “tumour” includes primary and metastatic tumours, as well as refractory tumours, solid or non-solid tumours. A further aspect of the present invention is a nucleic acid encoding the antibody as defined above or hybridizing with the above nucleic acid, or consisting of a correspondent degenerated sequence.

It is within the scope of the invention an expression vector encoding the antibody as defined above, preferably comprising the nucleic acid as defined above. It is within the scope of the invention a host cell comprising the nucleic acid as defined above, or the vector as defined above.

The terms “host cell”, “host cell line”, and “host cell culture” are used interchangeably and refer to cells into which an exogenous nucleic acid has been introduced, including the progeny of such cells. The host cells include “transformants” and “transformed cells,” which include the transformed primary cell and the progeny derived therefrom, without taking into account the number of steps. The progeny may be not completely identical in nucleic acid content to a parent cell, but may contain mutations. In the present invention mutant progenies are included, which have the same function or biological activity as that for which they have been screened or selected in the originally transformed cell. The nucleic acids of the invention can be used to transform a suitable mammalian host cell. Mammalian cells available as expression hosts are well known and include, for example, CHO and BHK cells. Prokaryotic hosts include, for example, E. coli, Pseudomonas, Bacillus, etc. Antibodies of the invention can be fused to additional amino acid residues, such as tags that facilitate their isolation. The term “vector”, as used in the present invention refers to a nucleic acid molecule capable of propagating another nucleic acid to which it is linked. The term includes the vector as a self-replicating nucleic acid structure as well as the vector incorporated into the genome of a host cell in which it was introduced. Certain vectors are capable of directing the expression of nucleic acids to which they are operably linked. In the present such vectors are referred to as “expression vectors.” Any suitable expression vector can be used, for example prokaryotic cloning vectors such as plasmids from E. coli, such as colE1, pCR1, pBR322, pMB9, pUC. Expression vectors suitable for expression in mammalian cells include derivatives of SV-40, adenovirus, retrovirus-derived DNA sequences. The expression vectors useful in the present invention contain at least one expression control sequence that is operatively linked to the sequence or fragment of DNA that must be expressed. It is a further of the invention a pharmaceutical composition comprising at least the antibody or a synthetic or recombinant fragment thereof as defined above and pharmaceutical acceptable excipients, preferably said composition being for use by parenteral administration, in particular intravenously. The composition comprises an effective amount of the antibody and/or recombinant or synthetic antigen binding fragments thereof. The pharmaceutical compositions are conventional in this field and can be produced by the skilled in the art just based on the common general knowledge. The formulations useful in therapy as described herein may e.g. comprise the antibody as described above, in a concentration from about 0.1 mg/ml to about 100 mg/ml, preferably from 0.1 to 10 mg/ml, more preferably from 0.1 to 5 mg/ml. In other formulations, the antibody concentration may be lower, e.g. at least 100 pg/ml. The antibody of the invention is administered to the patient in one or more treatments. Depending on the type and severity of the disease, a dosage of e.g. about 1 mg/kg to 20 mg/kg of the antibody may be administered, for example in one or more administrations, or by continuous infusion. The antibodies of the present invention may be administered in combination with other therapeutic agents, in particular with antibodies able to neutralize other receptors involved in tumour growth or angiogenesis. Any method of administration may be used to administer the antibody of the present invention, in particular, for example, the administration may be oral, intravenous, intraperitoneal, subcutaneous, or intramuscular. The antibody according to the present invention may also be administered as a conjugate, which binds specifically to the receptor and releases toxic substances. In particular embodiments, the pharmaceutical composition of the present invention can be administered in the form of single dosage (for example, tablet, capsule, bolus, etc.). For pharmaceutical applications, the composition may be in the form of a solution, for example, of an injectable solution, emulsion, suspension, or the like. The vehicle can be any vehicle suitable from the pharmaceutical point of view. Preferably, the vehicle used is capable of increasing the entry effectiveness of the molecules into the target cell. In the pharmaceutical composition according to the invention, the inhibitor or suppressor may be associated with other therapeutic agents, such as antagonists of other growth factor receptors involved in tumorigenesis or angiogenesis, such as VEGFR-2, EGFR, PDGFR, receptor kinase inhibitors, BRAF inhibitors, MEK inhibitors, immunomodulatory antibodies, anticancer agents, such as: bevacizumab, ramucirumab, aflibercept, sunitinib, pazopanib, sorafenib, cabozantinib, axitinib, regorafenib, nintedanib, lenvatinib, vemurafenib, dabrafenib, trametinib, chemotherapeutic agents such as methylating agents (temozolomide, dacarbazine), platinum compounds (cisplatin, carboplatin, oxaliplatin), taxanes (paclitaxel, nab-paclitaxel, docetaxel), fluoropyrimidines (5-fluorouracil, capecitabine), topoisomerase I inhibitors (irinotecan, topotecan), poly(ADP-ribose) polymerase inhibitors (PARP) (e.g., olaparib), etc. The pharmaceutical composition is chosen according to the demands of treatment. These pharmaceutical compositions according to the invention may be administered in the form of tablets, capsules, oral preparations, powders, granules, pills, liquid solutions for injection or infusion, suspensions, suppositories, preparations for inhalation. A reference for the formulations is the book by Remington (“Remington: The Science and Practice of Pharmacy”, Lippincott Williams & Wilkins, 2000). The skilled in the art will choose the form of administration and the effective dosages, by selecting suitable diluents, adjuvants and/or excipients.

The term “pharmaceutical composition” refers to a preparation that is in such a form as to permit to the biological activity of an active ingredient contained therein to be effective, and which contains no additional components which are unacceptably toxic to a subject to which the formulation may be administered. It is a further aspect of the invention a method for producing the antibody or a synthetic or recombinant fragment thereof as defined above, comprising the steps of culturing the host cell and purifying the antibody or a synthetic or recombinant fragment thereof from the cell culture.

In the context of the present invention the term “comprising” also includes the terms “having essentially” or “consisting essentially”.

In the present invention, the herein mentioned “protein(s)” also comprises the protein encoded by the corresponding orthologous or homologous genes, functional mutants, functional derivatives, functional fragments or analogues, isoform, splice variants thereof.

In the present invention “functional” is intended for example as “maintaining their activity”.

As used herein “fragments” refers to polypeptides having preferably a length of at least 10 amino acids, more preferably at least 15, at least 17 amino acids or at least 20 amino acids, even more preferably at least 25 amino acids or at least 37 or 40 amino acids, and more preferably of at least 50, or 100, or 150 or 200 or 250 or 300 or 350 or 400 or 450 or 500 amino acids.

The present invention will be described by means of non-limiting examples, referring to the following figures:

FIG. 1 Expression of IL-1R8 in human and mouse NK cells. a, b, IL-1R8 protein expression in human primary NK cells and other leukocytes (a) and NK cell maturation stages (b). MFI, mean fluorescence intensity. c, d, Il-1r8 mRNA expression in mouse primary NK cells and other leukocytes (c) and in sorted splenic NK cell subsets (d). *P<0.05, **P<0.01, ***P<0.001, one-way analysis of variance (ANOVA). Mean±s.e.m.

FIG. 2 NK cell differentiation and function in IL-1R8-deficient mice. a, b, NK cell frequency and absolute number among leukocytes in Il1r8^{+/+} and Il1r8^{-/-} mice. c, d, NK cell subsets (c) and KLRG1⁺ NK cells (d). e-g, IFNγ (e), granzyme B (f) and FasL (g) expression in stimulated NK cells. h, Splenic CD27^{low} NK cell frequency upon IL-18 in vivo depletion. i, IFNγ production by Il1r8^{+/+} and Il1r8^{-/-} NK cells upon co-culture with CpG-primed Il1r8^{+/+} dendritic cells and IL-18 blockade. j, IRAK4, S6 and JNK phosphorylation in NK cells upon stimulation with IL-18. k, RNA-seq analysis of resting and IL-18-activated NK cells. Differentially expressed (P<0.05) genes are shown. l, Correlation between IL-1R8 expression and IFNγ production in human peripheral blood NK cells. m, IL-1R8 expression and IFNγ production in human NK cells 7 days after transfection with control siRNA or IL-1R8-specific siRNA in duplicate. a-l, *P<0.05, **P<0.01, ***P<0.001 between selected relevant comparisons, two-tailed unpaired Student's t-test or Mann-Whitney U-test; k, r is Pearson's correlation coefficient. Mean±s.e.m.

FIG. 3 NK-cell-mediated protection against liver carcinogenesis and metastasis in IL-1R8-deficient mice. a, Macroscopic score of liver lesions in male Il1r8^{+/+} and Il1r8^{-/-} mice 6, 8, 10 and 12 months after diethylnitrosamine (DEN) injection. P values are given at the tops of graphs. b, Frequency and representative histological quantification of NK cell infiltrate in liver of tumour-bearing mice (original magnification 20×; scale bar, 100 μm). c, Frequency of IFNγ⁺ NK cells in liver of tumour-bearing mice. d, Macroscopic score of liver lesions in male mice upon NK cell depletion. e, Number of spontaneous lung metastases. f, NK cell frequency in the lungs of MN/MCA1 tumour-bearing mice. g, Number of lung metastases in MN/MCA1 tumour-bearing mice upon NK cell depletion. h, Number of liver metastases in MC38 colon carcinoma-bearing mice. i, j, Number of lung (i) and liver (j) metastases of Il1r8^{+/+} mice after adoptive transfer of Il1r8^{+/+} and Il1r8^{-/-} NK cells. a, d, Representative images of female livers are shown. a-j, Exact P values are given between selected relevant comparisons, two-tailed unpaired Student's t-test. Mean±s.e.m.

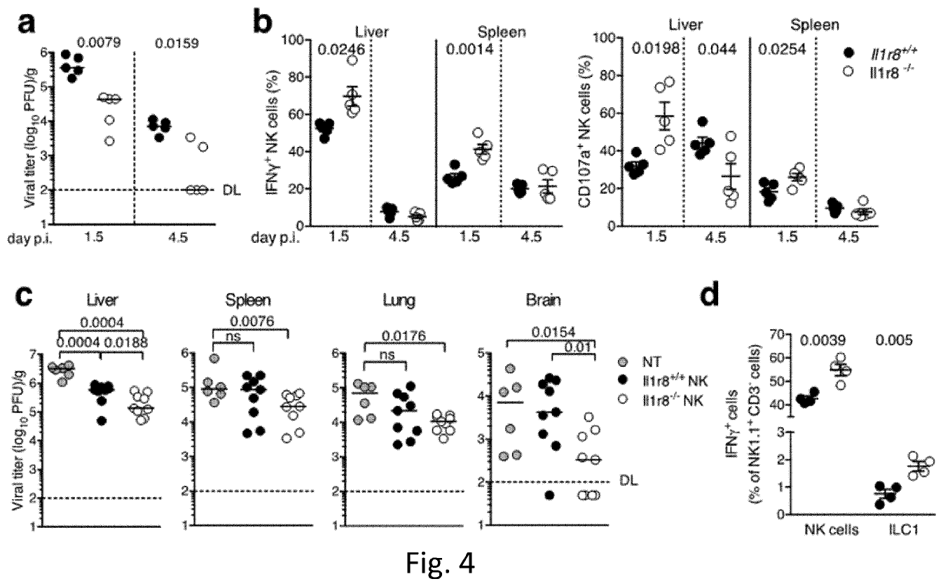

FIG. 4 NK-cell-mediated antiviral resistance in IL-1R8-deficient mice. a, Viral titre in livers of Il1r8^{+/+} and Il1r8^{-/-} infected mice. DL, detection limit. Day p.i., day post-infection. b, Frequency of IFNγ⁺ and CD107a⁺ NK cells of infected mice. c, Viral titres in newborn wild-type mice upon adoptive transfer of Il1r8^{+/+} and Il1r8^{-/-} NK cells (7 days after infection). d, Frequency of IFNγ⁺ cells in the liver of MCMV-infected mice. a-d, Exact P values are given, two-tailed Mann-Whitney U-test (a, c) or unpaired Student's t-test (b, d). Median (a, c); mean±s.e.m. (b, d).

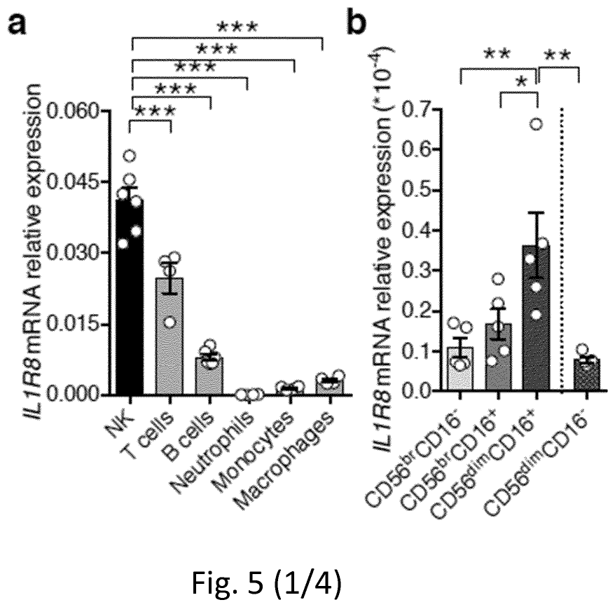

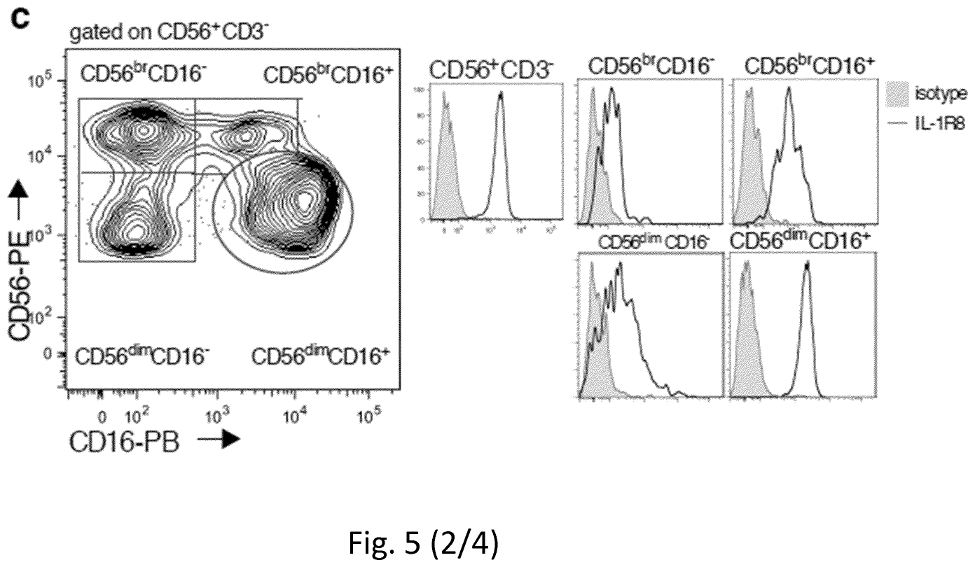

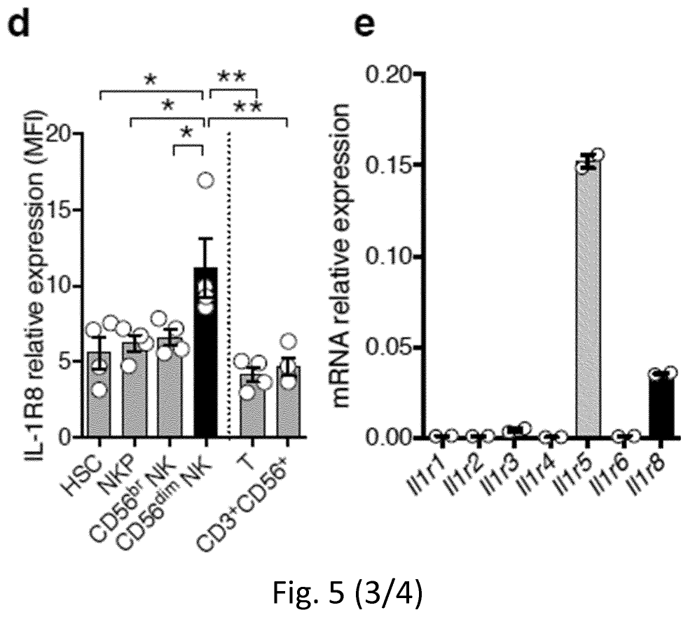

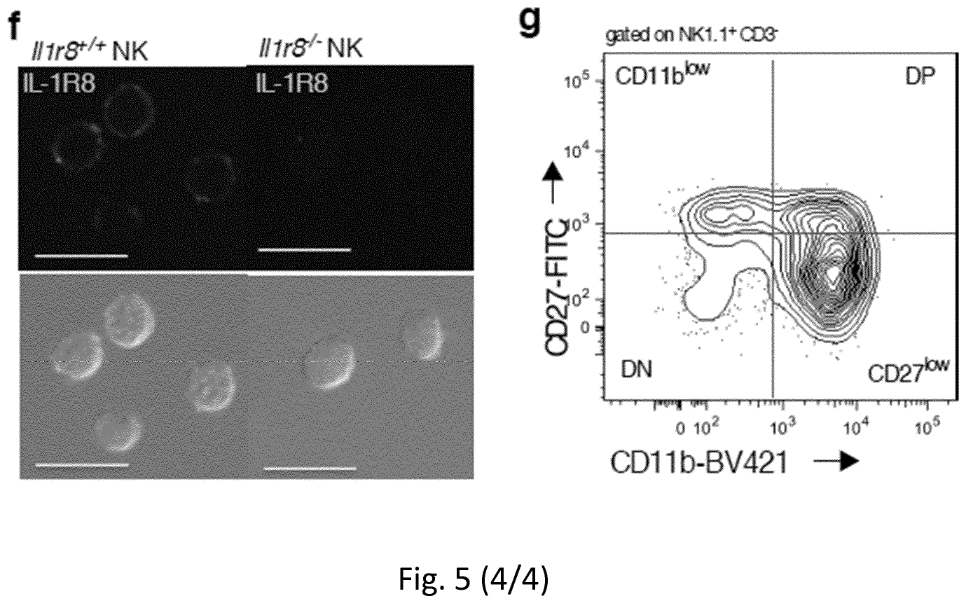

FIG. 5. Expression of IL-1R8 in human and mouse NK cells. a, b, II-1r8 mRNA (a) expression in human primary NK cells, compared with T and B cells, neutrophils, monocytes and in vitro-derived macrophages (a) and in human primary NK cell maturation stages (CD56^{br}CD16⁻, CD56^{br}CD16⁺, CD56^{dim}CD16⁺), and in the CD56^{dim}CD16⁻ subset (b). c, Representative plot of fluorescence-activated cell sorting of human NK cell subsets and histograms of IL-1R8 expression in NK cell subsets. d, IL-1R8 protein expression in human bone marrow precursors and mature cells. e, ILR family member (Il1r1, Il1r2, Il1r3, Il1r4, Il1r5, Il1r6, Il1r8) mRNA expression in mouse primary NK cells isolated from the spleen. f, IL-1R8 protein expression in mouse NK cells by confocal microscopy. Magnification bar, 10 μm. g, Representative plot of fluorescence-activated cell sorting of mouse NK cell subsets. a, b, d, *P<0.05, **P<0.01, ***P<0.001. One-way ANOVA. Mean±s.e.m. a, n=6 (NK and B cells) or n=4 donors; b, n=5 donors; d, n=4 donors; e, n=2 mice; f, representative images out of four collected per group. a, b, d-f, One experiment performed.

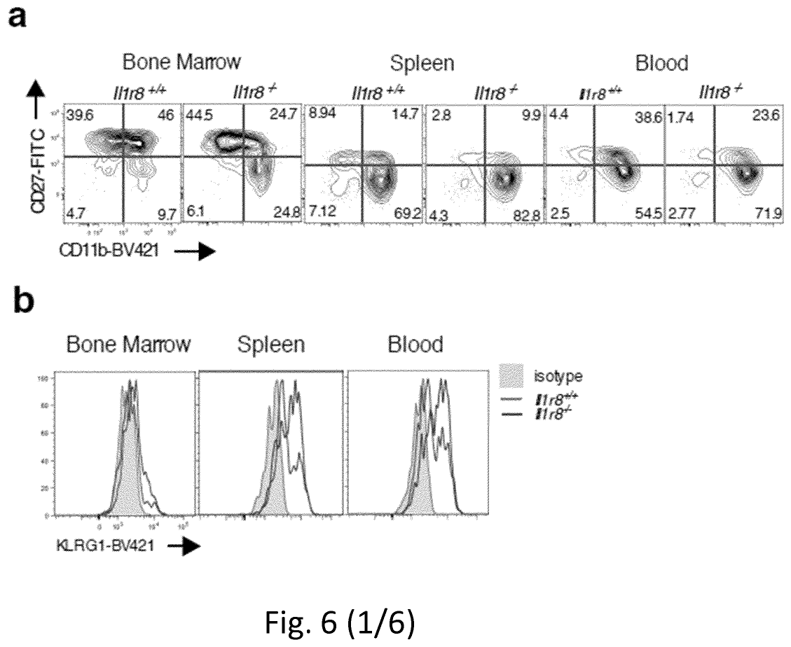

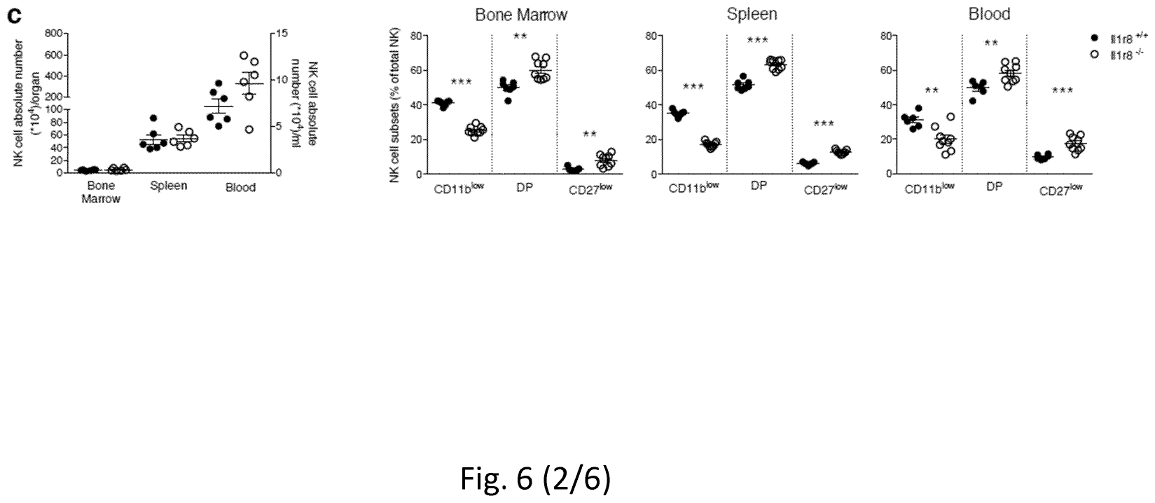

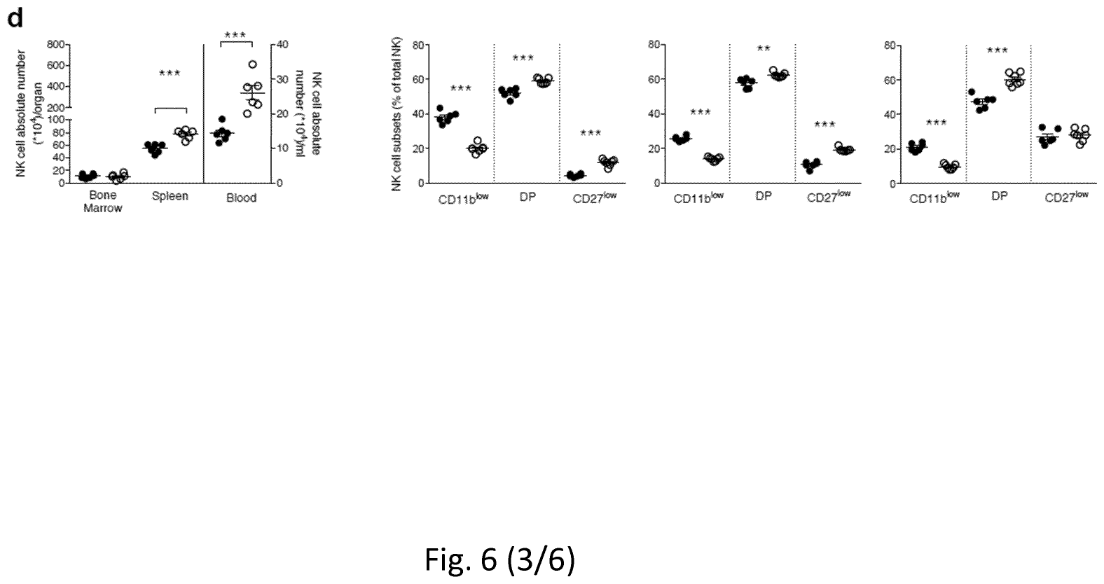

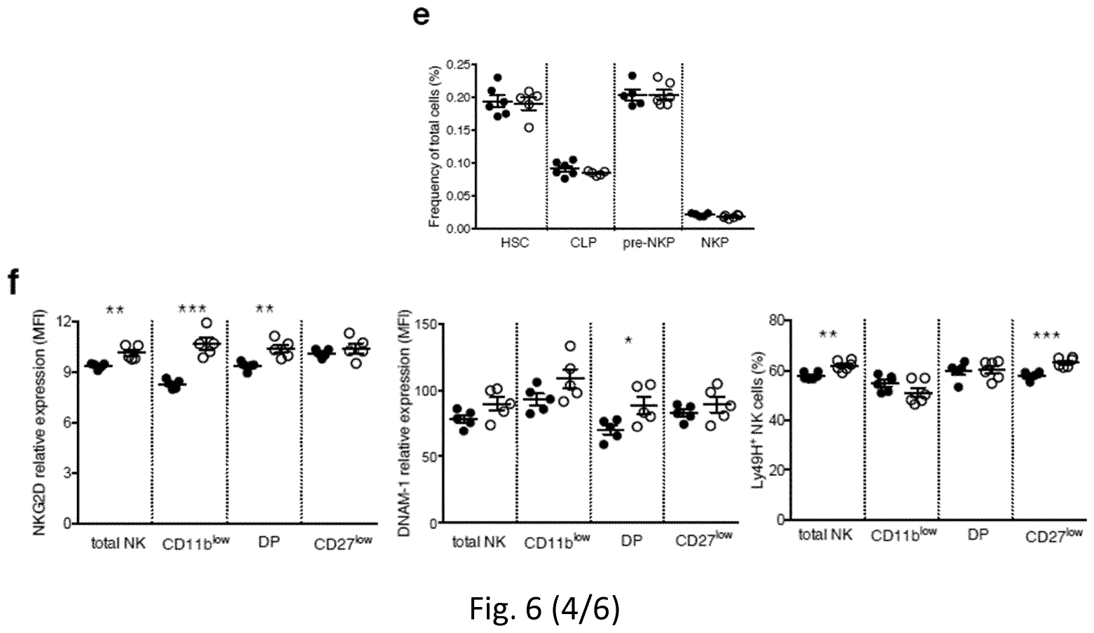

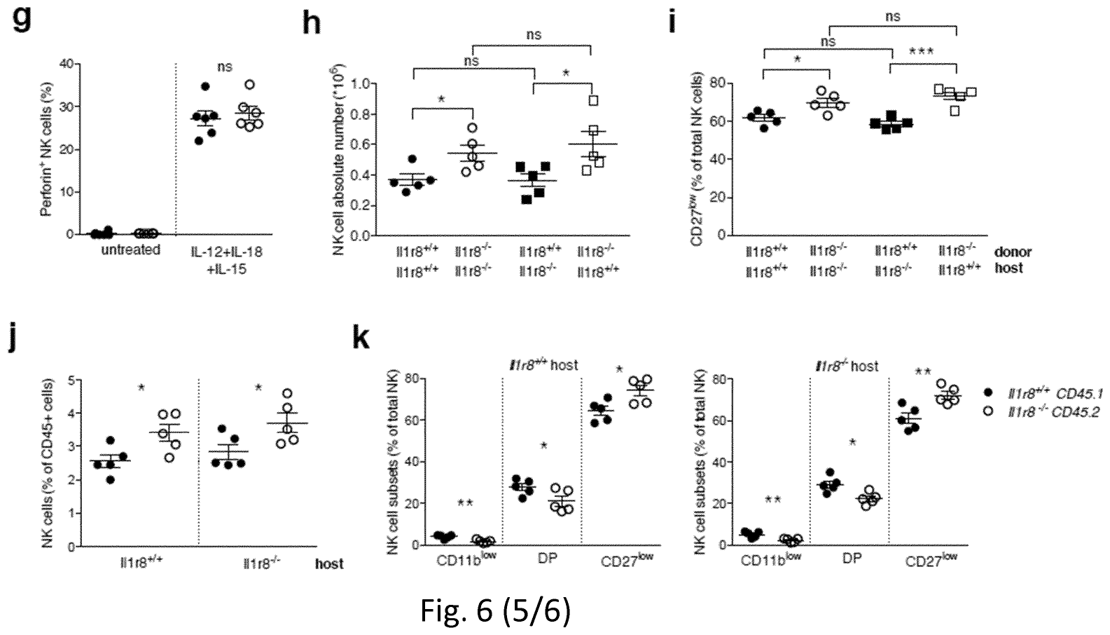

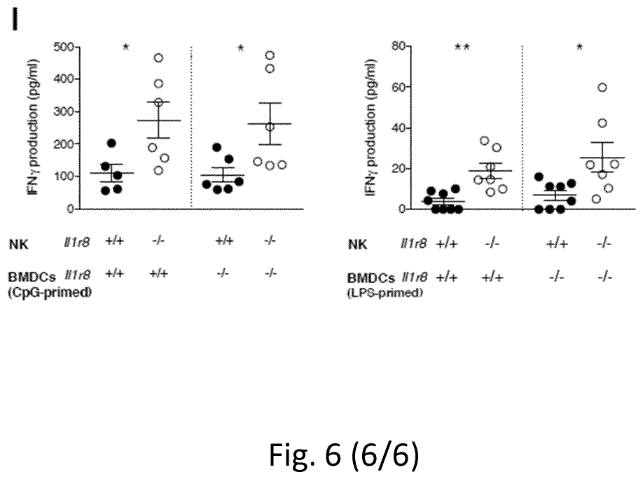

FIG. 6. Phenotypic analysis of Il1r8^{-/-} NK cells. a, b, Representative plot of fluorescence-activated cell sorting of mouse NK cell subsets in Il1r8^{+/+} and Il1r8^{-/-} mice (a) and histograms of KLRG1 expression in NK cells (b). c, d, NK absolute number and NK cell subsets (DN, CD11b^{low}, DP and CD27^{low}) in bone marrow, spleen and blood of Il1r8^{+/+} and Il1r8^{-/-} newborn mice at 2 (c) and 3 (d) weeks of age. e, Frequency of bone marrow precursors in Il1r8^{+/+} and Il1r8^{-/-} mice. f, NKG2D, DNAM-1 and LY49H expression in peripheral NK cells and NK cell subsets of Il1r8^{+/+} and Il1r8^{-/-} mice. g, Frequency of splenic Perforin⁺ NK cell subsets upon stimulation in Il1r8^{+/+} and Il1r8^{-/-} mice. h, i, Peripheral NK cell absolute number (h) and CD27^{low} NK cell frequency (i) in bone marrow chimaeric mice upon reconstitution (9 weeks). j, k, Peripheral NK cell (j) and NK cell subset (k) frequency in competitive chimaeric mice transplanted with 50% of Il1r8^{+/+} CD45.1 cells and 50% of Il1r8^{-/-} CD45.2 cells upon reconstitution (9 weeks). Upon reconstitution, a defective engraftment (12% instead of 50% engraftment) of Il1r8^{-/-} stem cells was observed in competitive conditions. l, IFNγ production by Il1r8^{+/+} and Il1r8^{-/-} NK cells upon co-culture with LPS- or CpG-primed Il1r8^{+/+} and Il1r8^{-/-} dendritic cells. c-l, *P<0.05, **P<0.01, ***P<0.001 between selected relevant comparisons, two-tailed unpaired Student's t-test. Centre values and error bars, mean±s.e.m. At least five animals per group were used. c, d, Three pooled experiments; e-l, one experiment was performed.

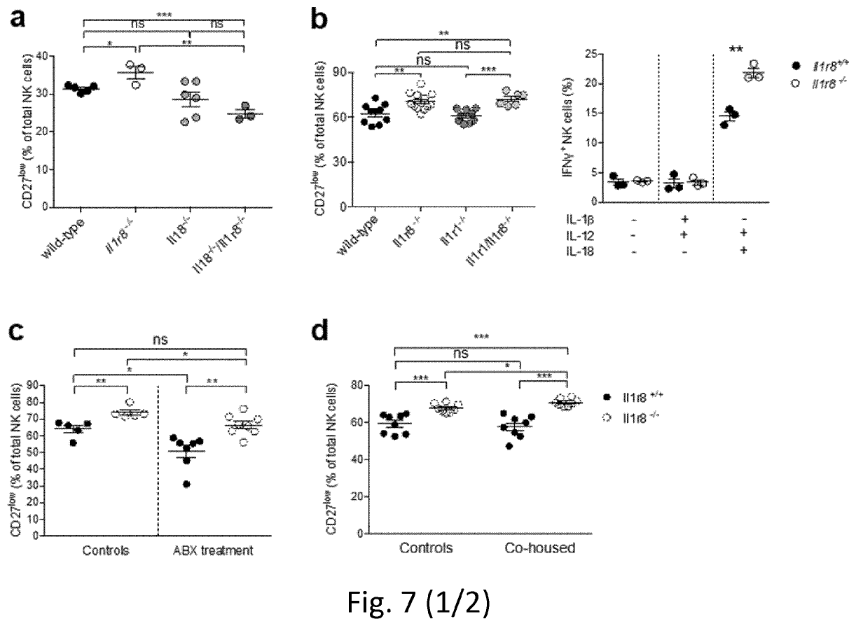

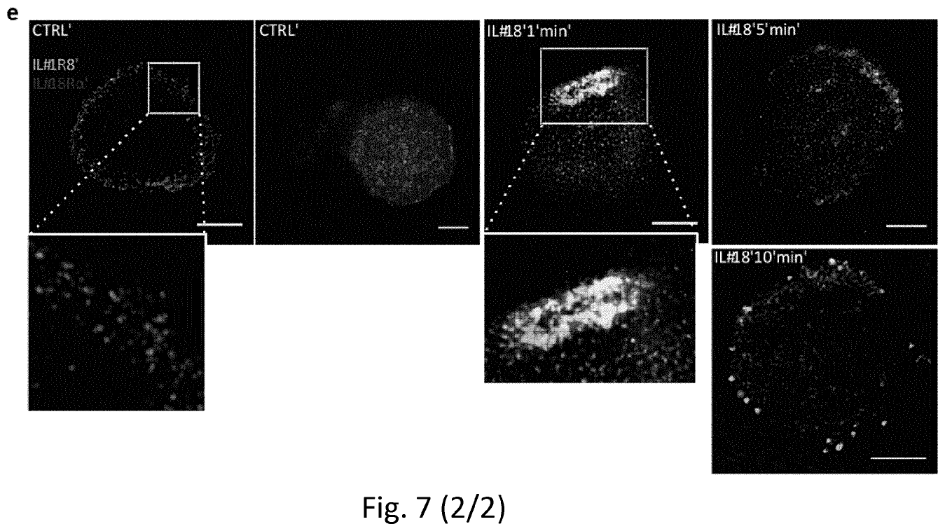

FIG. 7. Mechanism of IL-1R8-dependent regulation of NK cells. a, Splenic CD27^{low} NK cell frequency in wild-type, Il1r8^{-/-}, Il1r8^{-/-} and Il1r8^{-/-}/Il1r8^{-/-} mice. b, Peripheral CD27^{low} NK cell frequency in wild-type, Il1r8^{-/-}, Il1r8^{-/-} and Il1r8^{-/-}/Il1r8^{-/-} mice (left) and IFNγ production by splenic NK cells after IL-12 and IL-1β or IL-18 stimulation (right). c, d, Splenic CD27^{low} NK cell frequency in Il1r8^{+/+} and Il1r8^{-/-} mice upon commensal flora depletion (c) and breeding in co-housing conditions (d). e, STED microscopy of human NK cells stimulated with IL-18. Magnification bar, 2 μm. a-d, *P<0.05, **P<0.01, ***P<0.001 between selected relevant comparisons, two-tailed unpaired Student's t-test; Centre values and error bars, mean±s.e.m. a, n=3, 5, or 6 mice; at least five animals per group were used (b-d). a-d, One experiment was performed. e, Representative images out of three collected from two donors.

FIG. 8. RNA-seq analysis of Il1r8^{+/+} and Il1r8^{-/-} NK cells. Metascape analysis of enriched gene pathways of resting and IL-18-activated Il1r8^{+/+} and Il1r8^{-/-} NK cells. See also data deposited in the NCBI Gene Expression Omnibus under accession number GSE105043.

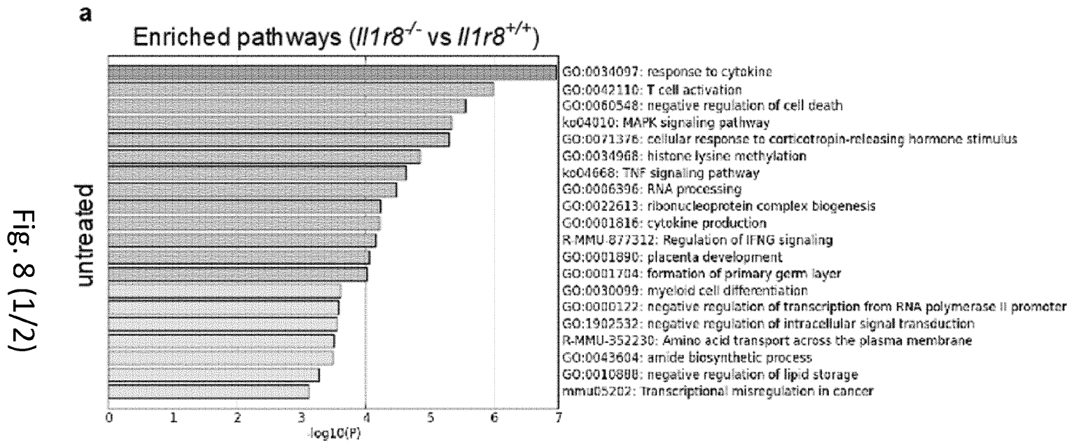

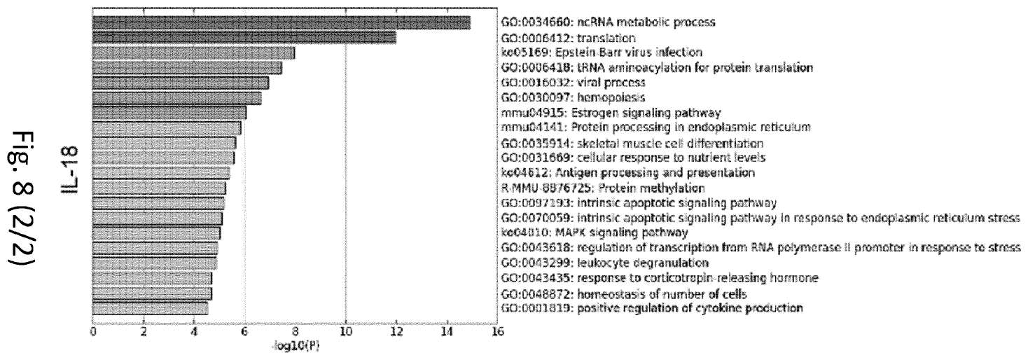

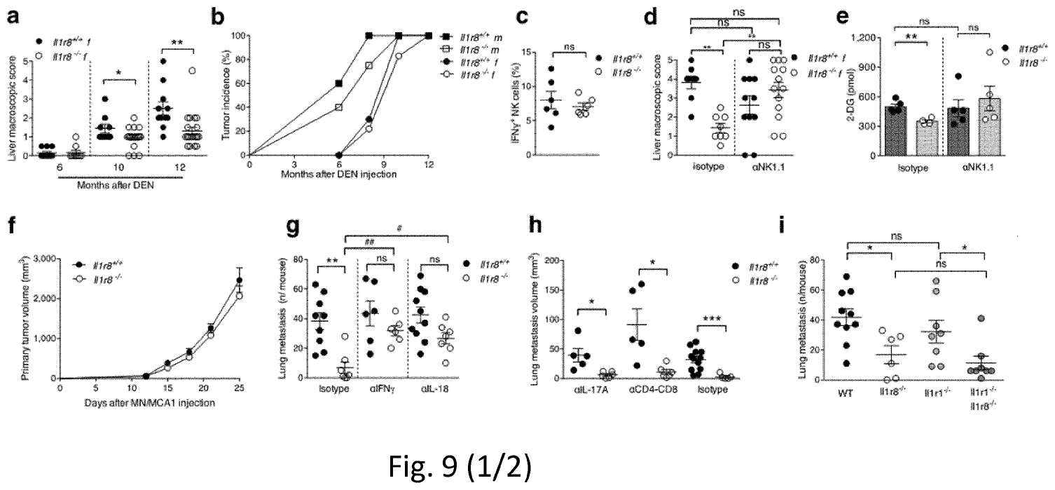

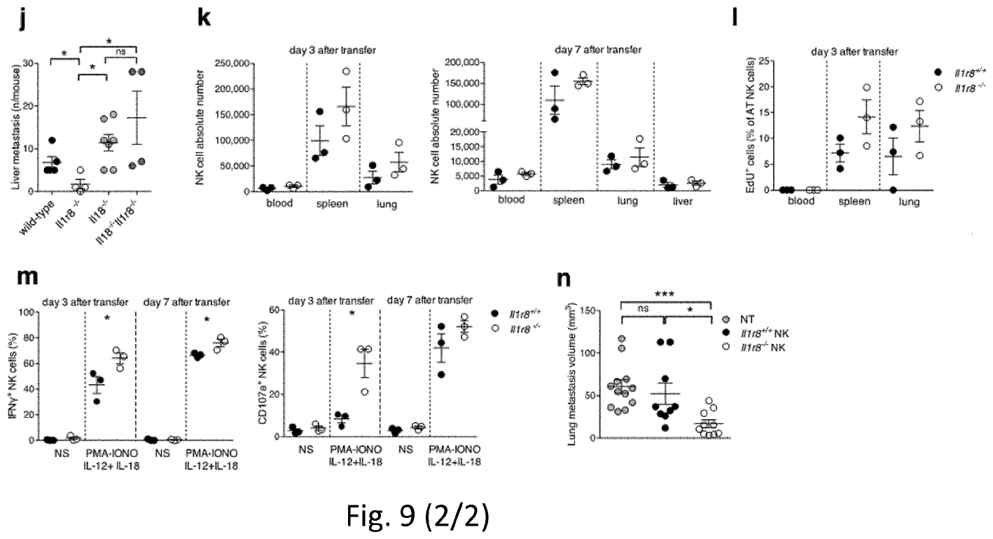

FIG. 9. NK-cell-mediated resistance to hepatocellular carcinoma and metastasis in IL-1R8-deficient mice. a, Macroscopic score of liver lesions in female Il1r8^{+/+} and Il1r8^{-/-} mice 6, 10 and 12 months after diethylnitrosamine (DEN) injection. b, Incidence of hepatocellular carcinoma in Il1r8^{+/+} and Il1r8^{-/-} female and male mice. c, Frequency of IFNγ⁺ NK cells in spleen of Il1r8^{+/+} and Il1r8^{-/-} tumour-bearing mice. d, Macroscopic score of liver lesions in female Il1r8^{+/+} and Il1r8^{-/-} mice upon NK cell depletion. e, 2-Deoxyglucosone (2-DG) quantification in lungs of Il1r8^{+/+} and Il1r8^{-/-} tumour-bearing mice upon NK cell depletion. f, Primary tumour growth in Il1r8^{+/+} and Il1r8^{-/-} mice (25 days after MN/MCA1 cell line injection). g, Number of lung metastases in Il1r8^{+/+} and Il1r8^{-/-} MN/MCA1 sarcoma-bearing mice upon IFNγ or IL-18 neutralization. h, Volume of lung metastases in Il1r8^{+/+} and Il1r8^{-/-} MN/MCA1-bearing mice upon depletion of IL-17A or CD4⁺/CD8⁺ cells. i, Number of lung metastases in Il1r8^{+/+} and Il1r8^{-/-}, Il1r1^{-/-}, Il1r1^{-/-}/Il1r8^{-/-} MN/MCA1-bearing mice. j, Number of liver metastases in Il1r8⁺⁺, Il1r8^{-/-}, Il1r8^{-/-}, Il1r8^{-/-}/Il1r8^{-/-} MC38 colon carcinoma-bearing mice. k, Il1r8^{+/+} and Il1r8^{-/-} NK cell absolute number 3 or 7 days after adoptive transfer. l, In vivo Il1r8^{+/+} and Il1r8^{-/-} NK cell proliferation 3 days after adoptive transfer. m, Ex vivo IFNγ production and degranulation upon 4 h stimulation with PMA-ionomycin, IL-12 and IL-18 in adoptively transferred Il1r8^{+/+} and Il1r8^{-/-} NK cells. n, Volume of lung metastases in Il1r8^{+/+} MN/MCA1 sarcoma-bearing mice after adoptive transfer of Il1r8^{+/+} and Il1r8^{-/-} NK cells. a, c-e, g-j, m-n, *P<0.05, **P<0.01, ***P<0.001 between selected relevant comparisons, two-tailed unpaired Student's t-test or Mann-Whitney U-test. #P<0.05, ##P<0.01, Kruskal-Wallis and Dunn's multiple comparison test. Centre values and error bars, mean±s.e.m. a, n=9, 10, 11, 18, 21 mice; b, n=8-21 mice; c, n=6 mice; d, n=10, 12, 13 mice; e, n=4 (Il1r8^{-/-} isotype) or n=5; f, n=10; g, n=6, 7, 9, 10 mice; h, n=5, 6, 12 mice; i, n=6, 8, 10 mice; j, n=4, 5, 7 mice; k, l, m, n=3 mice; n, n=9, 10, 12 mice. Representative experiment out of three (a, b), 2 (d), 6 (f), or one (c, e, g-n) experiments performed.

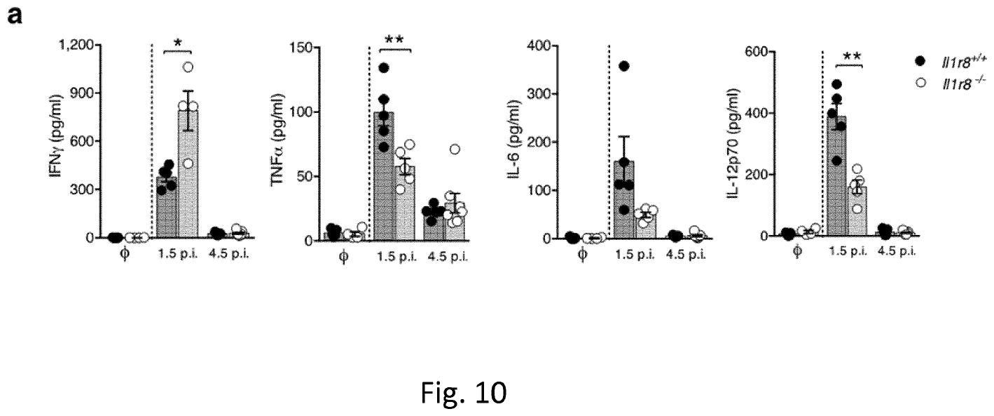

FIG. 10. NK-cell-mediated antiviral resistance in IL-1R8-deficient mice. Cytokine serum levels in Il1r8^{+/+} and Il1r8^{-/-} infected mice (1.5 and 4.5 days after infection). *P<0.05, **P<0.01, ***P<0.001, unpaired Student's t-test. Centre values and error bars, mean±s.e.m.; n=5 mice. One experiment was performed.

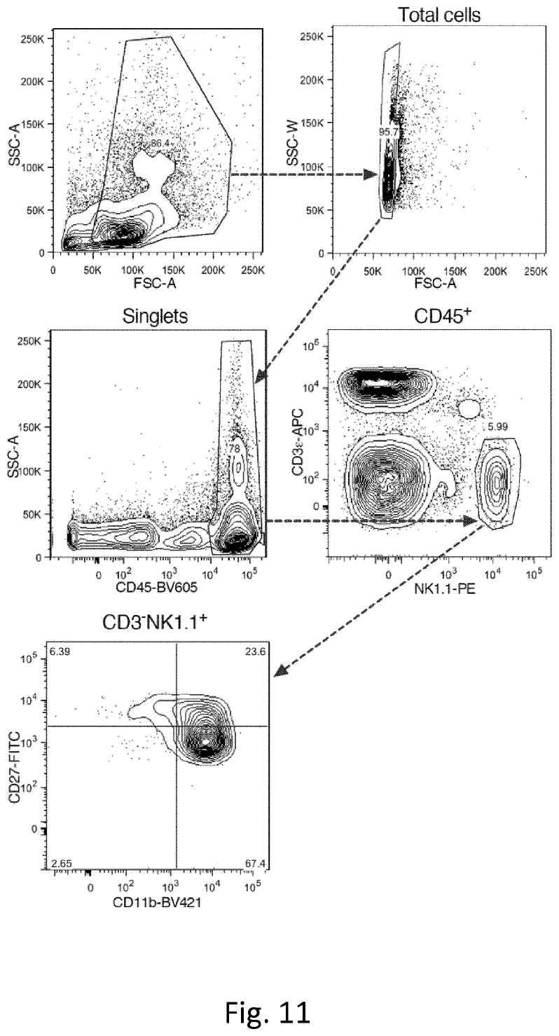

FIG. 11. Murine splenic NK cell gating strategy, used for FACS analysis and NK cell sorting.

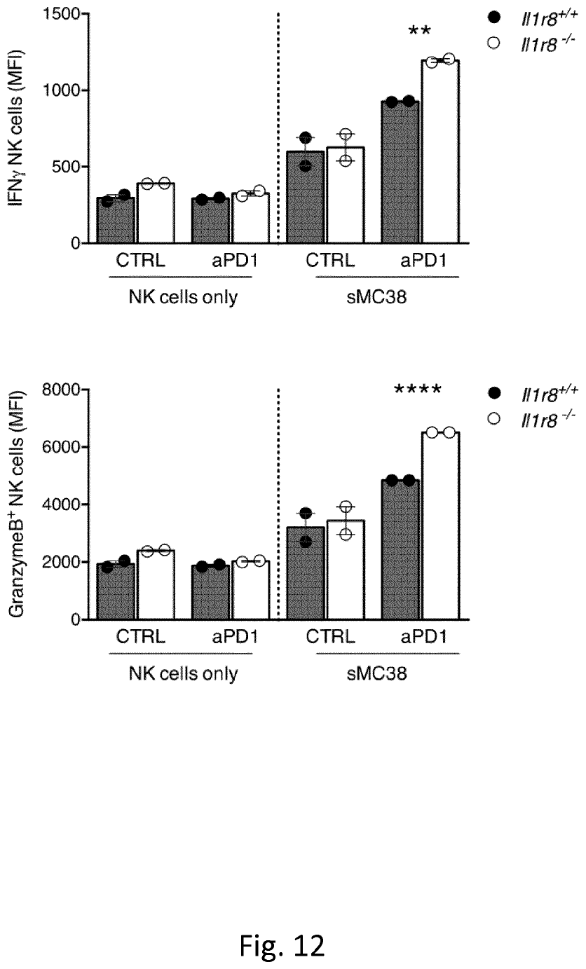

FIG. 12. NK cell functional activation by anti-PD-1. IFNγ (upper panel) and Granzyme B (lower panel) intracellular staining in NK cells in basal conditions (cultured alone in the presence of a control antibody (CTRL)) or after activation by culture with the target (stimulated MC38 colorectal cancer cells) and anti-PD-1 antibody (aPD-1). NK cells were purified and treated as described in methods and analyzed by flow cytometry. MFI=mean fluorescence intensity. Student's T test. N=2 mice.

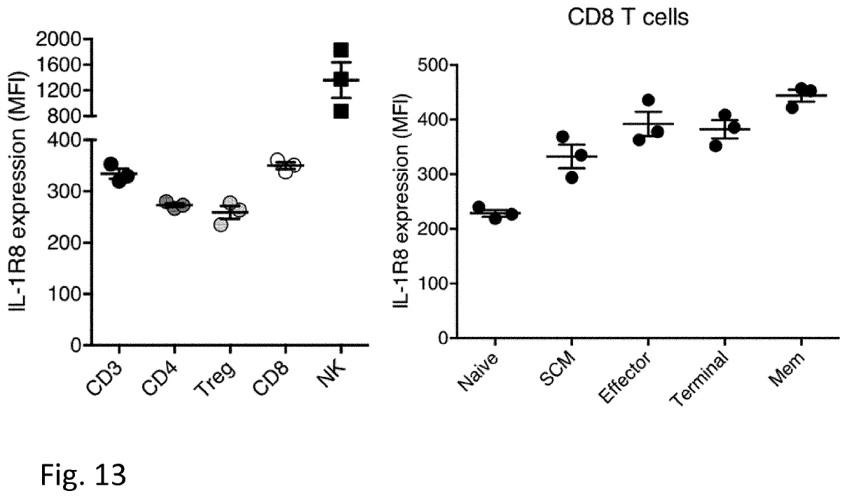

FIG. 13. IL-1R8 expression in human lymphocytes. IL-1R8 expression was analysed by flow cytometry. CD8+ T cell subsets were defined based on the following gating strategy: a) Naïve T cell subset: CD3+, CD8+, CCR7+, CD45RO−, b) Stem Cell Memory (SCM) T cell subset: CD3+, CD8+, CCR7+, CD45RO−, CD95+; c) Effector T cell subset: CD3+, CD8+, CCR7−, CD45RO+; d) Terminal Effector T cell subset: CCR7−, CD45RO−; Central memory (Mem): CD3+, CD8+, CCR7+, CD45RO+. MFI=mean fluorescence intensity.

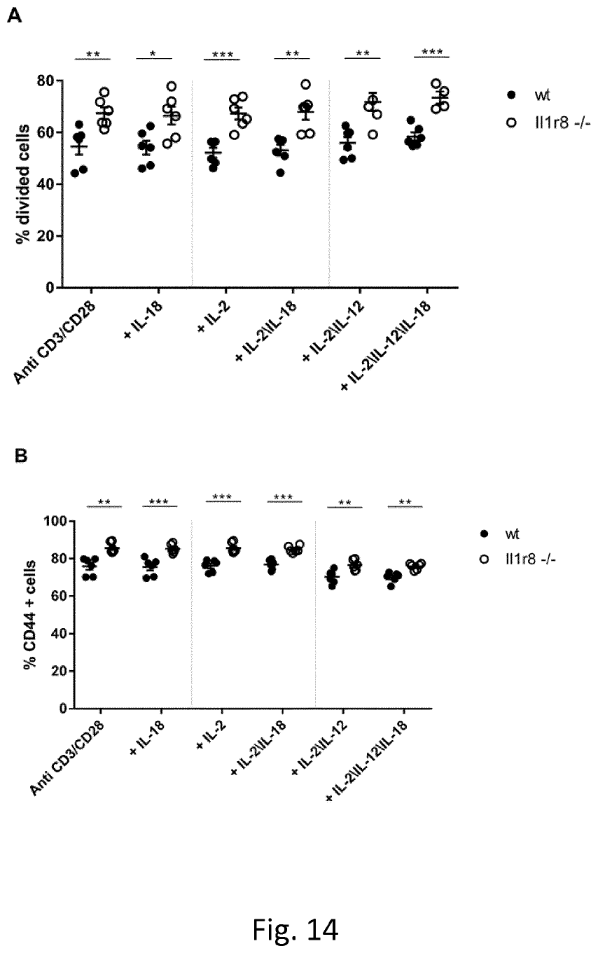

FIG. 14. Mouse CD8+ T cell proliferation and maturation. A) CD8+ T cell proliferation was assessed as described in methods and reported as percentage of divided cells. B) Expression of the maturation marker CD44 after activation. Student's T test. N=6 mice.

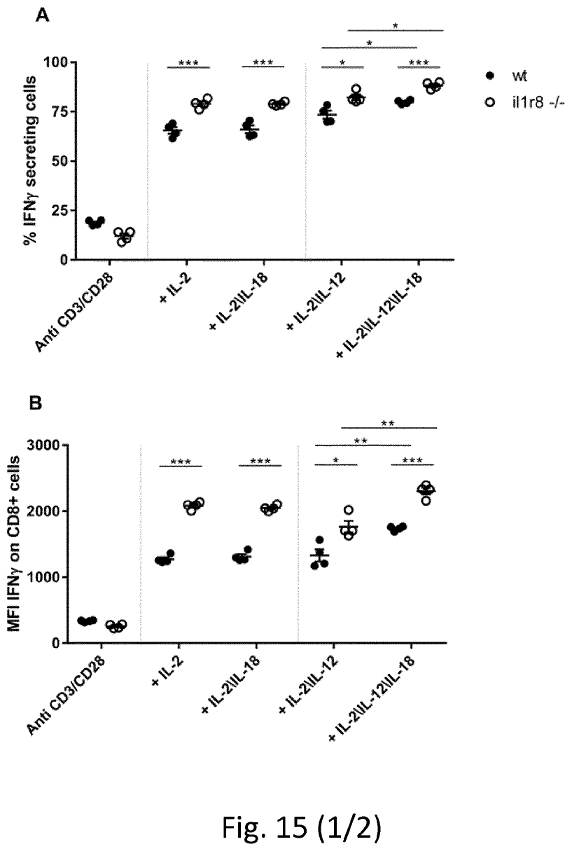

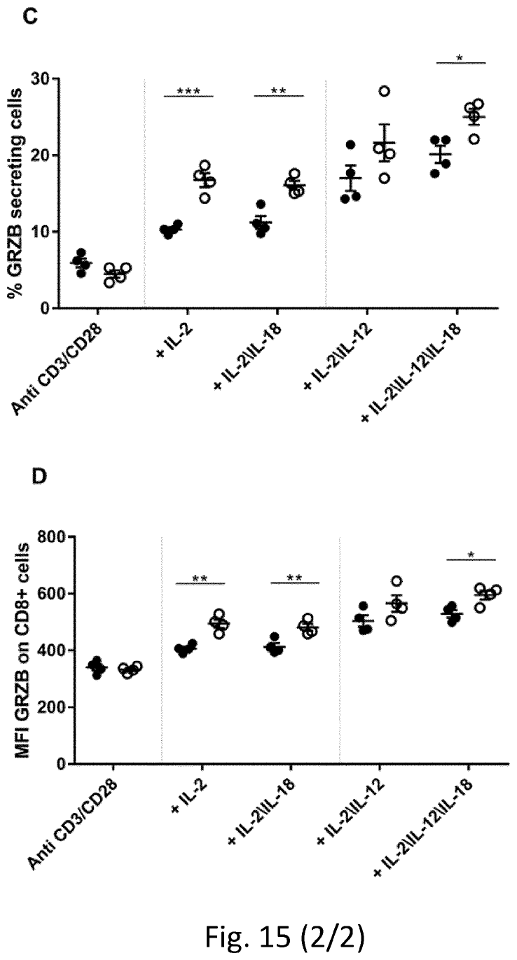

FIG. 15. CD8+ T cell activation. Expression of IFNγ (A, B) and Granzyme B (C, D) after stimulation with anti-CD3/CD28 and cytokines (11-2, IL-12, IL-18). Results are reported as percentage of positive cells or mean fluorescence intensity (MFI). Student's T test. N=4 mice.

TABLE 1
Serum cytokine and liver enzyme levels in hepatocellular carcinoma-bearing mice

| | 6 months after DEN | | | 8-10 months after DEN | | | 12 months after DEN | | |
|---|---|---|---|---|---|---|---|---|---|
| Cytokine | Il1r8^{+/+} | Il1r8^{-/-} | p | Il1r8^{+/+} | Il1r8^{-/-} | p | Il1r8^{+/+} | Il1r8^{-/-} | p |
| pg/mL | n = 4-5* | n = 5 | value | n = 7-10* | n = 9-10* | value | n = 3-5* | n = 3-5* | value |
| IL-23 | 173.1 ± 29.12 | 247.3 ± 15.16 | 0.05 | 187.7 ± 13.47 | 343.4 ± 66.29 | 0.04 | 103.7 ± 26.72 | 138.6 ± 37.51 | 0.47 |
| IL-12p70 | 277.6 ± 44.49 | 358.4 ± 12.44 | 0.12 | 293 ± 16.31 | 357.2 ± 34.77 | 0.13 | 152 ± 20.14 | 164.9 ± 15.22 | 0.62 |
| IL-17A | 69.98 ± 9.88 | 95.03 ± 6.44 | 0.07 | 56.41 ± 7.46 | 102.4 ± 19.01 | 0.04 | 38.13 ± 10.39 | 45.05 ± 8.78 | 0.62 |
| IFNγ | 295 ± 72.78 | 385.4 ± 48.6 | 0.32 | 357.5 ± 57.63 | 593.2 ± 84.33 | 0.05 | 195.4 ± 65.29 | 243.3 ± 104 | 0.72 |
| IL-6 | 90.37 ± 6.45 | 67.23 ± 9.79 | 0.08 | 126.9 ± 19.52 | 69.64 ± 6.93 | 0.01 | 61.24 ± 18.05 | 42.28 ± 12.17 | 0.44 |
| IL-1β | 91.99 ± 5.23 | 58.68 ± 7.29 | 0.006 | 142.4 ± 28.24 | 60.35 ± 4.42 | 0.01 | 47.66 ± 14.08 | 29.81 ± 7.66 | 0.31 |
| TNFα | 163.5 ± 7.16 | 92.06 ± 21.04 | 0.01 | 194.6 ± 28.03 | 100.1 ± 14.24 | 0.008 | 94.77 ± 14.24 | 57.45 ± 14.51 | 0.13 |
| CCL2 | 32.51 ±1.54 | 24.1 ± 5.64 | 0.19 | 43.97 ± 7.25 | 25.42 ± 1.37 | 0.02 | 28.1 ± 4.99 | 19.72 ± 1.23 | 0.14 |
| CXCL1 | 197.6 ± 8.85 | 142.5 ± 20.93 | 0.04 | 183.4 ± 17.75 | 123.7 ± 10.5 | 0.01 | 105.6 ± 6.49 | 77.86 ± 9.64 | 0.04 |
| Liver | | | | | | | | | |
| enzymes** | | | | | | | | | |
| ALT | 142.5 ± 52.5 | 0.00 ± 0.00 | 0.004 | 111.7 ± 70.77*** | 60.0 ± 35.0*** | 0.32 | 0.00 ± 0.00 | 0.00 ± 0.00 | NA |
| AST | 159.6 ± 39.79 | 101.0 ± 1.87 | 0.18 | 134.0 ± 15.28*** | 97.0 ± 8.0*** | 0.06 | 105.0 ± 25.45 | 89.0 ± 5.1 | 0.55 |

*Samples with no detectable levels were not included in the analysis.
**levels are U/L.
***n = 5, 8 months after DEN

### EXAMPLE 1

Materials and Methods

Animals

All female and male mice used were on a C57BL/6J genetic background and were 8-12 weeks old, unless otherwise specified. Wild-type mice were obtained from Charles River Laboratories, Calco, Italy, or were littermates of Il1r8^{-/-} mice. IL-1R8-deficient mice were generated as described³¹. Il1r1^{-/-} mice were purchased from The Jackson Laboratory, Bar Harbour, Me., USA. All colonies were housed and bred in the SPF animal facility of Humanitas Clinical and Research Center in individually ventilated cages. Il1r1^{-/-}/Il1r8^{-/-} mice were generated by crossing Il1r1^{-/-} and Il1r8^{-/-} mice. Il1r8^{-/-}/Il1r8^{-/-} were generated by crossing Il1r8^{-/-} and Il1r8^{-/-} mice. Mice were randomized on the basis of sex, age and weight. Procedures involving animal handling and care conformed to protocols approved by the Humanitas Clinical and Research Center (Rozzano, Milan, Italy) in compliance with national (D.L. N.116, G.U., suppl. 40, 18 Feb. 1992 and N. 26, G.U. Mar. 4, 2014) and international law and policies (EEC Council Directive 2010/63/EU, OJ L 276/33, 22 Sep. 2010; National Institutes of Health Guide for the Care and Use of Laboratory Animals, US National Research Council, 2011). The study was approved by the Italian Ministry of Health (approval number 43/2012-B, issued on the 8 Feb. 2012, and number 828/2015-PR, issued on the 7 Aug. 2015). All efforts were made to minimize the number of animals used and their suffering. In most in vivo experiments, the investigators were unaware of the genotype of the experimental groups.

Human Primary Cells

Human peripheral mononuclear cells were isolated from peripheral blood of healthy donors, upon approval by the Humanitas Research Hospital Ethical Committee. Peripheral mononuclear cells were obtained through a Ficoll density gradient centrifugation (GE Healthcare Biosciences). NK cells were then purified by a negative selection, using a magnetic cell-sorting technique according to the protocols given by the manufacturer (EasySep Human NK Cell Enrichment Kit, Stem Cell Technology). Human monocytes were obtained from peripheral blood of healthy donors by two-step gradient centrifugation, first by Ficoll and then by Percoll (65% iso-osmotic; Pharmacia, Uppsala, Sweden). Residual T and B cells were removed from the monocyte fraction by plastic adherence. Monocytes were cultured in RPMI-1640 medium supplemented with 10% fetal bovine serum (FBS), 1% L-glutamine, 1% penicillin/streptomycin and 100 ng ml⁻¹ M-CSF (Peprotech) for 7 days to generate resting macrophages. T and B cells were obtained from peripheral blood of healthy donors using RosetteSep Human T Cell Enrichment Cocktail and RosetteSep Human B Cell Enrichment Cocktail (Stem Cell Technology), following the manufacturer's instructions. Neutrophils were enriched from Ficoll-isolated granulocytes, using an EasySep Human Neutrophil Enrichment Kit (StemCell Technologies), according to the manufacturer's instructions. To analyse pluripotent haematopoietic stem cells and NK cell precursors, human bone marrow mononuclear cells were collected from Humanitas Biobank, upon approval by the Humanitas Research Hospital Ethical Committee (authorization 1516, issued on 26 Feb. 2016). Frozen samples were thawed and vitality was assessed by trypan blue and Aqua LIVE/Dead-405 nm staining (Invitrogen), before flow cytometry analysis. Informed consent was obtained from all participants.

Fluorescence-Activated Cell Sorting Analysis

Single-cell suspensions of bone marrow, blood, spleen, lung and liver were obtained and stained. A representative NK cell gating strategy is reported in FIG. 11A. Foxp3/Transcription Factor Staining Buffer Set (eBioscience) was used for intracellular staining of granzyme B and perforin. Cytofix/Cytoperm (BD Biosciences) was used for intracellular staining of IFNγ. Liver ILC1 were identified as NK1.1⁺ CD3⁻ CD49a⁺ CD49b cells. Formalin 4% and methanol 100% were used for intracellular staining of IRAK4, pIRAK4, pS6 and JNK. The following mouse antibodies were used: CD45-BV605, -BV650 or -PerCp-Cy5.5 (clone 30-F11); CD45.1-BV650 (clone A20); CD45.2-APC, -BV421 (clone 104); CD3e-PerCP-Cy5.5 or -APC (clone 145-2C11); CD19-PerCP-Cy5.5, -eFluor450 (clone 1D3); NK1.1-PE, -APC, -eFluor450 or -Biotin (clone PK136); CD11b-BV421, -BV450, -BV785 (clone M1/70); CD27-FITC or -APC-eFluor780 (clone LG.7F9); CD4-FITC (clone RM 4-5); CD8-PE (clone 53-6.7); KLRG-1-BV421 (clone 2F1); NKG2D-APC (clone CX5); DNAM-1-APC (clone 10E5); Ly49H-PECF594 (clone 3D10); Granzyme B-PE (clone NGZB); Perforin-PE (clone eBioOMAK-D); IFNγ-Alexa700 or -APC (clone XMG1.2); CD107a-Alexa647 (clone 1D4B); FasL-APC (clone MFL3); Lineage Cell Detection Cocktail-Biotin; Sca-1-FITC (clone D7); CD117-PE or -Biotin (clone 3C11); CD127-eFluor450 (clone A7R34); CD135-APC or -Biotin (clone A2F10.1); CD244-PE (clone 2B4); CD122-PE-CF594 (clone TM-Beta1); CD49b-PE-Cy7 or Biotin (clone DX5), CD49a-APC (clone Ha31/8), from BD Bioscience, eBioscience, BioLegend or Miltenyi Biotec. The following human antibodies were used: CD56-PE (clone CMSSB); CD3-FITC (clone UCHT1); CD16-Pacific Blue (clone 3G8); CD34-PE-Vio770 (clone AC136); CD117-BV605 (clone 104D2); NKp46-BV786 (clone 9E2/NKp46); CD45-PerCP (clone 2D1); CD19-APC-H7 (clone SJ25C1); CD14-APC-H7 (clone M5E2); CD66b-APC-Vio770 (clone REA306), from BD Bioscience, eBioscience or Miltenyi Biotec. Biotinylated anti-hSIGIRR (R&D Systems) and streptavidin Alexa Fluor 647 (Invitrogen) were used to stain IL-1R8 in human cells. Human NKT cells were detected using PE-CD1d tetramers loaded with aGalCer (ProImmune, Oxford, UK). Antibodies to detect protein phosphorylation were as follows: p-IRAK4 Thr345/Ser346 (clone D6D7), IRAK4, p-S6-Alexa647 Ser235/236 (clone D57.2.2E); p-SAPK/JNK Thr183/Tyr185 (clone 81E11), from Cell Signaling Technology. A goat anti-rabbit Alexa Fluor 647 secondary antibody (Invitrogen) was used to stain p-IRAK4, IRAK4 and p-SAPK/JNK. Results are reported as mean fluorescence intensity normalized on isotype control or fluorescence minus one. Cell viability was determined by Aqua LIVE/Dead-405 nm staining (Invitrogen) or Fixable Viability Dye (FVD) eFluor 780 (eBioscience); negative cells were considered viable. Cells were analysed on an LSR Fortessa or FACSVerse (BD Bioscience). Data were analysed with FlowJo software (Treestar).

Quantitative PCR

Total RNA was extracted using Trizol reagent (Invitrogen) following the manufacturer's recommendations. RNA was further purified using an miRNeasy RNA isolation kit (Qiagen) or Direct-zol RNA MiniPrep Plus (Zymo Research). cDNA was synthesized by reverse transcription using a High Capacity cDNA Archive Kit (Applied Biosystems) and quantitative real-time PCR was performed using SybrGreen PCR Master Mix (Applied Biosystems) in a CFX96 Touch Real-Time PCR Detection System (Bio-Rad). PCR reactions were performed with 10 ng of DNA. Data were analysed with the 2^{(-ΔCT)} method. Data were normalized on the basis of GAPDH, β-actin or 18S expression, as indicated, determined in the same sample. Analysis of all samples was performed in duplicate. Primers were designed according to the published sequences and listed as follows: s18/S18: forward 5′-ACT TTC GAT GGT AGT CGC CGT-3′ (SEQ ID NO:5), reverse 5′-CCT TGG ATG TGG TAG CCG TTT-3′ (SEQ ID NO:6); Gapdh/GAPDH: forward 5′-GCA AAG TGG AGA TTG TTG CCA T-3′ (SEQ ID NO:7), reverse 5′-CCT TGA CTG TGC CGT TGA ATT T-3′ (SEQ ID NO:8); βactin/βACTIN: forward 5′-CCC AAG GCC AAC CGC GAG AAG AT-3′ (SEQ ID NO:9), reverse 5′-GTC CCG GCC AGC CAG GTC CAG-3′ (SEQ ID NO: 10); il1r8: forward 5′-AGA GGT CCC AGA AGA GCC AT-3′ (SEQ ID NO: 11), reverse 5′-AAG CAA CTT CTC TGC CAA GG-3′ (SEQ ID NO: 12); IL1R8: forward 5′-ATG TCA AGT GCC GTC TCA ACG-3′ (SEQ ID NO:13), reverse 5′-GCT GCG GCT TTA GGA TGA AGT-3′ (SEQ ID NO:14); il1r1: forward 5′-TGC TGT CGC TGG AGA TTG AC-3′ (SEQ ID NO: 15), reverse 5′-TGG AGT AAG AGG ACA CTT GCG AA-3′ (SEQ ID NO:16); il1r2: forward 5′-AGT GTG CCC TGA CCT GAA AGA-3′ (SEQ ID NO:17), reverse 5′-TCC AAG AGT ATG GCG CCC T-3′ (SEQ ID NO:18); il1r3: forward 5′-GGC TGG CCC GAT AAG GAT-3′ (SEQ ID NO:19), reverse 5′-GTC CCC AGT CAT CAC AGC G-3′ (SEQ ID NO:20); il1r4: forward 5′-GAA TGG GAC TTT GGG CTT TG-3′ (SEQ ID NO:21), reverse 5′-GAC CCC AGG ACG ATT TAC TGC-3′ (SEQ ID NO:22); il1r5: forward 5′-GCT CGC CCA GAG TCA CTT TT-3′ (SEQ ID NO:23), reverse 5′-GCG ACG ATC ATT TCC GAC TT-3′ (SEQ ID NO:24); il1r6: forward 5′-GCT TTT CGT GGC AGC AGA TAC-3′ (SEQ ID NO:25), reverse 5′-CAG ATT TAC TGC CCC GTT TGT T-3′ (SEQ ID NO:26); 16S: forward 5′-AGA GTT TGA TCC TGG CTC AG-3′ (SEQ ID NO:27), reverse 5′-GGC TGC TGG CAC GTA GTT AG-3′ (SEQ ID NO:28).

Purification of Mouse Leukocytes

Splenic NK cells and bone marrow neutrophils were enriched by MACS® according to the manufacturer's instructions (Miltenyi Biotec). Purity of NK cells was about 90% as determined by fluorescence-activated cell sorting. The purity of neutrophils was ≥97.5%. NK cells were stained (CD45-BV650, NK1.1-PE, CD3e-APC, CD11b-BV421, CD27-FITC) and sorted on a FACSAria cell sorter (BD Bioscience) to obtain high-purity NK cells and NK cell populations (CD11b^{low}CD27^{low}, CD11b^{low}CD27^{high}, CD11b^{high}CD27^{high} and CD11b^{high}CD27^{low}). Splenic B and T lymphocytes were stained (CD45-PerCP, CD3e-APC, CD4-FITC, CD8-PE, CD19-eFluor450) and sorted. The purity of each population was ≥98%. Resulting cells were processed for mRNA extraction or used for adoptive transfer or co-culture experiments. In vitro-derived macrophages were obtained from bone marrow total cells. Bone marrow cells were cultured in RPMI-1640 medium supplemented with 10% FBS, 1% L-glutamine, 1% penicillin/streptomycin and 100 ng ml⁻¹ M-CSF (Peprotech) for 7 days to generate resting macrophages. Bone marrow cells were cultured in RPMI-1640 medium supplemented with 10% FBS, 1% L-glutamine, 1% penicillin/streptomycin and 20 ng ml⁻¹ GM-CSF (Peprotech) for 7 days to generate dendritic cells.

Confocal Microscopy

Mouse splenic NK cells were enriched by magnetic cell sorting, left to adhere on poly-D-lysine (Sigma-Aldrich) coated coverslips, fixed with 4% PFA, permeabilized with 0.1% Triton X-100 and incubated with blocking buffer (5% normal donkey serum (Sigma-Aldrich), 2% BSA, 0.05% Tween). Cells were then stained with biotin-conjugated goat polyclonal anti-SIGIRR antibody or biotin-conjugated normal goat IgG as control (both R&D Systems) (10 μg ml⁻¹) followed by Alexa Fluor 488-conjugated donkey anti-goat IgG antibody (Molecular Probes) and 4′,6-diamidino-2-phenylindole (DAPI) (Invitrogen). Coverslips were mounted with the antifade medium FluorPreserve Reagent (EMD Millipore) and analysed with an Olympus Fluoview FV1000 laser scanning confocal microscope with a 40× oil immersion lens (numerical aperture 1.3).

Stimulated Emission Depletion (STED) Microscopy

Human NK cells were enriched and left to adhere on poly-D-lysine (Sigma-Aldrich)-coated coverslips, stimulated with IL-18 (50 ng ml⁻¹; 1 min, 5 min, 10 min), fixed with 4% PFA, incubated with 5% normal donkey serum (Sigma-Aldrich), 2% BSA, 0.05% Tween in PBS2+ (pH 7.4) (blocking buffer), and then with biotin-conjugated goat polyclonal anti-human IL-1R8 antibody or biotin-conjugated normal goat IgG (all from R&D Systems) and mouse monoclonal anti-IL-18Rα (Clone 70625; R&D Systems) or mouse IgG1 (Invitrogen), all diluted at 5 μg ml⁻¹ in blocking buffer, followed by Alexa Fluor 488-conjugated donkey anti-goat IgG antibody and Alexa Fluor 555 donkey anti-mouse IgG antibody (both from Molecular Probes). Mowiol was used as mounting medium. STED xyz images were acquired in a unidirectional mode with a Leica SP8 STED3X confocal microscope system. Alexa Fluor 488 was excited with a 488 nm argon laser and emission collected from 505 to 550 nm applying a gating between 0.4 and 7 ns to avoid collection of reflection and autofluorescence. Alexa Fluor 555 was excited with a 555/547 nm-tuned white light laser and emission collected from 580 to 620 nm. Line sequential acquisition was applied to avoid fluorescence overlap. The 660 nm CW-depletion laser (80% of power) was used for both excitations. Images were acquired with Leica HC PL APO 100×/1.40 numerical aperture oil STED White objective at 572.3 milli absorption units (mAU). CW-STED and gated CW-STED were applied to Alexa Fluor 488 and Alexa Fluor 555, respectively. Collected images were de-convolved with Huygens Professional software.

3′-mRNA Sequencing and Analysis

Splenic NK cells (from six mice per genotype and pooled in pairs) were purified as described above and stimulated with IL-18 (MBL) (20 ng ml⁻¹ for 4 h). RNA was prepared as described above. A QuantSeq 3′mRNA-seq Library Prep Kit for Illumina (Lexogen) was used to generate libraries, which were sequenced on the NextSeq (Illumina; 75 bp PE). The fastq sequence files were assessed using the fastqc program. The reads were first trimmed using bbduk in the bbmap suite of software³² to remove the first 12 bases and a contaminant kmer discovery length of 13 was used for contaminant removal. Regions of length 20 or above with average quality of less than 10 were trimmed from the end of the read. The reads were then trimmed to remove trailing polyG and polyA runs using cutadapt³³ and the quality of the remaining reads reassessed with fastqc. The trimmed reads were aligned to the mm10 genomic reference and reads assigned to features in the mm10 annotation using the STAR program³⁴. Differential expression analysis used the generalized linear model functions in the R/bioconductor³⁵ edgeR package³⁶ with TMM normalization. Gene set analysis used the romer³⁷ function in the R/bioconductor package limma³⁸. Metascape (http://metascape.org) was used to enrich genes for Gene Ontology biological processes, KEGG Pathway and Reactome Gene Sets.

Measurement of Cytokines

A BD Cytometric Bead Array (CBA) mouse inflammation kit (BD) or Duoset ELISA kits (R&D Systems) were used to measure cytokines.

In Vitro Functional Assays

Total mouse splenocytes or enriched mouse or human NK cells were cultured in RPMI-1640 medium supplemented with 10% FBS 1% L-glutamine, 1% penicillin/streptomycin and treated with IL-2, IL-12, IL-15 (Peprotech), IL-18 (MBL), IL-13 (Peprotech) and PMA-Ionomycin (Sigma-Aldrich), as specified. FasL expression was evaluated upon treatment for 45 min with IL-18 (50 ng ml⁻¹), IL-15 (50 ng ml⁻¹), IL-2 (20 ng ml⁻¹) and IL-12 (10 ng ml⁻¹). IFNγ production was analysed upon 16 h of treatment with IL-12 (20 ng ml⁻¹) and IL-18 (20 ng ml⁻¹) or IL-1β (20 ng ml⁻¹), by intracellular staining using a BD Cytofix/Cytoperm Fixation/Permeabilization Kit, following the manufacturer's instructions, or by ELISA. Granzyme B and perforin intracellular staining was performed upon 18 h of stimulation with IL-12 (10 ng ml⁻¹), IL-15 (10 ng ml⁻¹) and IL-18 (50 ng ml⁻¹⁻¹), using a Foxp3/Transcription Factor Staining Buffer Set (eBioscience). CD107a-Alexa Fluor 647 antibody was added during the 4 h culture and analysed by flow cytometry. BD GolgiPlug (containing Brefeldin) and BD GolgiStop (containing Monensin) were added 4 h before intracellular staining. PMA (50 ng ml⁻¹) and ionomycin (1 μg ml⁻¹) were added 4 h before intracellular staining, when specified.

NK-dendritic-cell co-culture experiments were performed as previously described³⁹. Dendritic cells were treated with LPS from Escherichia coli 055:B5 (Sigma-Aldrich; 1 μg ml⁻¹) or CpG ODN 1826 (Invivogen; 3 μg ml⁻¹) and with anti-mIL-18 neutralizing antibody (BioXCell, Clone YIGIF74-1G7; 5 μg ml⁻¹) or Rat Isotype Control (BioXCell, Clone 2A3).

IFNγ and CD107a expression upon viral infection was analysed by flow cytometry upon 4 h treatment with BD GolgiPlug, BD GolgiStop and IL-2 (500 U ml⁻¹).

Phosphorylation of IRAK4, S6 and JNK was analysed upon 15-30 min stimulation with IL-18 (10 ng ml⁻¹).

Human Primary NK Cell Transfection

Human NK cells were enriched from peripheral blood of healthy donors and transfected with Dharmacon Acell siRNA (GE Healthcare) using Accell delivery medium (GE Healthcare), following the manufacturer's instructions. SIGIRR-specific siRNA (1 μM) (On-Target Plus; Dharmacon, GE Healthcare) comprised 250 nM of the four following antisense sequences: I,

(SEQ ID NO: 1)
AGU UUC GCG AGC CGA GAU CUU;
(SEQ ID NO: 2)
II, UAC CAG AGC AGC ACG UUG AUU;
(SEQ ID NO: 3)
III, UGA CCC AGG AGU ACU CGU GUU;
(SEQ ID NO: 4)
IV, CUU CCC GUC GUU UAU CUC CUU.
(all 5′ to 3′)

Generation of Bone Marrow Chimaeras

Il1r8^{-/-} and Il1r8^{+/+} mice were lethally irradiated with a total dose of 900 cGy. Two hours later, mice were injected in the retro-orbital plexus with 4×10⁶ nucleated bone marrow cells obtained by flushing of the cavity of freshly dissected femurs from wild-type or Il1r8^{-/-} donors. Competitive bone marrow chimaeric mice were generated by reconstituting recipient mice with 50% CD45.1 Il1r8^{+/+} and 50% CD45.2 Il1r8^{-/-} bone marrow cells. Recipient mice received gentamycin (0.8 mg ml⁻¹ in drinking water) starting 10 days before irradiation and for 2 weeks after irradiation. NK cells of chimaeric mice were analysed 8 weeks after bone marrow transplantation.

Depletion and Blocking Experiments

Mice were treated intraperitoneally with 200 μg of specific mAbs (mouse anti-NK1.1, clone PK136; mouse isotype Control, clone C1.18.4; rat anti-mIL-18, clone YIGIF74-1G7; rat isotype Control, clone 2A3; rat anti-IFNγ, clone XMG1.2; rat IgG1 HRPN; mouse anti-IL-17A, clone 17F3; mouse isotype Control, clone MOPC-21; rat anti-CD4/CD8, clone GK1.5/YTS; rat isotype Control, clone LTF-2 (all from BioXCell)) and then with 100 μg once (anti-NK1.1) or three times (anti-IL-18, anti-IFNγ, anti-IL-17A, anti-CD4/CD8) a week for the entire duration of the experiment.

Microflora Depletion

Six-week-old mice were treated every day for 5 weeks by oral gavage with a cocktail of antibiotics (ampicillin (Pfizer) 10 mg ml⁻¹, vancomycin (PharmaTech Italia) 10 mg ml⁻¹, metronidazol (Societa Prodotti Antibiotici) 5 mg ml⁻¹ and neomycin (Sigma-Aldrich) 10 mg ml⁻¹). Control mice were treated with drinking water. A gavage volume of 10 ml/kg (body weight) was delivered with a stainless-steel tube without prior sedation of mice. DNA was isolated from bacterial faecal pellets with a PowerSoil DNA Isolation Kit (MO BIO Laboratories) and quantified by spectrophotometry at 260 nm. PCR was performed with 10 ng of DNA using SybrGreen PCR Master Mix (Applied Biosystems) in a CFX96 Touch Real-Time PCR Detection System (Bio-Rad). Data were analysed with the 2^{(-ΔCT)} method (Applied Biosystems, Real-Time PCR Applications Guide).

Cancer Models

Mice were injected intraperitoneally with 25 mg/kg (body weight) of diethylnitrosamine (Sigma) at 15 days of age. They were euthanized 6, 8, 10 or 12 months later, to analyse liver cancer. Liver cancer score was based on the number and volume of lesions (0: no lesions; 1: lesion number <3, or lesion dimension <3 mm; 2: lesion number <5, or lesion dimension <5 mm; 3: lesion number <10, or lesion dimension <10 mm; 4: lesion number <15, or lesion dimension <10 mm; 5: lesion number >15, or lesion dimension >10 mm). Lung metastasis experiments were performed injecting intramuscularly the 3-MCA-derived mycoplasma-free sarcoma cell line MN/MCA1 (10⁵ cells per mouse in 100 μl PBS)⁴⁰. Primary tumour growth was monitored twice a week, and lung metastases were assessed by in vivo imaging and by macroscopic counting at the time of being euthanized 25 days after injection. Liver metastases were generated by injecting intrasplenically 1.5×10⁵ mycoplasma-free colon carcinoma cells (MC38)²¹. Mice were euthanized 12 days after injection and liver metastases were counted macroscopically. MC38 cells were received from ATCC just before use. MN/MCA1 cells were authenticated morphologically by microscopy in vitro and by histology ex vivo. Tumour size limit at which mice were euthanized was based on major diameter (not more than 2 cm).

Viral Infections

Mice were injected intravenously with 5×10⁵ plaque-forming units of the tissue-culture-grown virus in PBS. Bacterial artificial chromosome-derived MCMV strain MW97.01 has been previously shown to be biologically equivalent to MCMV strain Smith (VR-1399) and is hereafter referred to as wild-type MCMV⁴¹. Mice were euthanized 1.5 and 4.5 days after infection and viral titre was assessed by plaque assay, as previously described^{42,43}. Newborn mice were infected intraperitoneally with 2,000 plaque-forming units of the MCMV strain MW97.01 and euthanized at day 7 after infection. Viral titre was assessed by plaque assay, as previously described^{42,43}.

Adoptive Transfer

One million Il1r8^{+/+} or Il1r8^{-/-} sorted NK cells were injected intravenously in wild-type adult mice 5 h before MN/MCA or MC38 injection, or intraperitoneally in newborn mice 48 h after MCMV injection. Adoptively transferred NK cell engraftment, proliferative capacity and functionality (IFNγ production and degranulation after ex vivo stimulation) were assessed 3 and 7 days after injection.

In Vivo Proliferation

In vivo proliferation was measured using a Click-iT Edu Flow Cytometry Assay Kit (Invitrogen). Edu was injected intraperitoneally (0.5 mg per mouse), mice were euthanized 24 h later and cells were stained following the manufacturer's instructions and analysed by flow cytometry.

Immunohistochemistry

Frozen liver tissues were cut at 8 mm and then fixed with 4% PFA. Endogenous peroxidases were blocked with 0.03% H₂O₂ for 5 min and unspecific binding sites were blocked with PBS+1% FBS for 1 h. Tissues were stained with polyclonal goat anti-mouse NKp46/NCR1 (R&D Systems) and a Goat-on-Rodent HRP polymer kit (GHP516, Biocare Medical) was used as secondary antibody. Reactions were developed with 3,3′-diaminobenzidine (Biocare Medical) and then slides were counterstained with haematoxylin. Slides were mounted with eukitt (Sigma-Aldrich). Images at 20× magnification were analysed with cell\hat{ }F software (Olympus).

In Vivo Imaging

After feeding with AIN-76A alfalfa-free diet (Mucedola, Italy) for 2 weeks to reduce fluorescence background, mice were intravenously injected with XenoLight RediJect 2-deoxyglucosone (PerkinElmer) and 24 h later 2-deoxyglucosone fluorescence was measured using a Fluorescence Molecular Tomography system (FMT 2000, Perkin Elmer). Acquired images were subsequently analysed with TrueQuant 3.1 analysis software (Perkin Elmer).

Statistical Analysis

For animal studies, sample size was defined on the basis of past experience on cancer and infection models, to detect differences of 20% or greater between the groups (10% significance level and 80% power). Values were expressed as mean±s.e.m. or median of biological replicates, as specified. One-way ANOVA or a Kruskal-Wallis test were used to compare multiple groups. A two-sided unpaired Student's t-test was used to compare unmatched groups with Gaussian distribution and Welch's correction was applied in cases of significantly different variance. A Mann-Whitney U-test was used in cases of non-Gaussian distribution. A ROUT test was applied to exclude outliers. P<0.05 was considered significant. Statistics were calculated with GraphPad Prism version 6, GraphPad Software.

Statistics and Reproducibility

FIG. 1a, n=4 (B cells), n=5 (NKT cells), n=9 (T cells), n=10 (NK cells) donors; FIG. 1b, n=5 donors; FIG. 1c, n=8 (NK cells) or n=4 (T cells) or n=3 (other leukocytes) mice; FIG. 1d, n=5 mice. FIG. 1b, Representative experiment out of six performed. FIG. 1a, c, d, one experiment performed.

FIG. 2a, b, n=8 or n=7 (spleen, Il1r8^{+/+} liver) or n=6 (Il1r8^{-/-} liver) mice; FIG. 2c, n=6 mice; FIG. 2d, n=9 (Il1r8^{+/+}) or n=6 (Il1r8^{-/-}) mice; FIG. 2e, n=5 mice; FIG. 2f, n=6 mice; FIG. 2g, n=4 mice; FIG. 2h, n=5 mice; FIG. 2i, n=10 wells; FIG. 2j, n=4 (IRAK4), n=6 or n=5 (S6 Il1r8^{-/-}) or n=7 (JNK Il1r8^{-/-}) mice; FIG. 2k, n=3 mice; FIG. 2i, n=9 healthy donors; FIG. 2m, n=4 healthy donors. Representative experiments out of three (FIG. 2a, b), five (FIG. 2c), two (FIG. 2d, j), four (FIG. 2e) performed. FIG. 2f-m, one experiment performed.

FIG. 3a, n=8, 10, 11, 13, 14 mice; FIG. 3b, c, n=6 mice; FIG. 3d, n=10, 12, 13 mice; FIG. 3e, n=10, 11 mice; FIG. 3f, n=5, 6, 7 mice; FIG. 3g, n=9, 10 mice; FIG. 3h, n=5, 6 mice; FIG. 3i, n=9, 10 or 12 mice; FIG. 3j, n=6 mice. Representative experiments out of 6 (FIG. 3e), 3 (FIG. 3a), 2 (FIG. 3d, f, g, h, i). FIG. 3b, c, j, one experiment performed.

FIG. 4a, b, n=5 mice; FIG. 4c, n=6, n=9 mice; FIG. 4d, n=4 mice. FIG. 4a, two experiments were performed. FIG. 4b-d, one experiment was performed.

Results

IL-1R8 is widely expressed¹⁰. However, inventors found strikingly high levels of IL-1R8 mRNA and protein in human NK cells, compared with other circulating leukocytes and monocyte-derived macrophages (FIG. 1a and FIG. 5a). IL1R8 mRNA levels increased during NK cell maturation¹¹ (FIG. 5b) and surface protein expression mirrored transcript levels (FIG. 1b and FIG. 5c). IL-1R8 expression was detected at a low level in bone marrow pluripotent haematopoietic stem cells and NK cell precursors, and was selectively upregulated in mature NK cells but not in CD3+ lymphocytes (FIG. 5d).

Mouse NK cells expressed significantly higher levels of Il1r8 mRNA compared with other leukocytes (FIG. 1c) and other ILRs (FIG. 5e, f). In line with the results obtained in human NK cells, the Il1r8 mRNA level increased during the four-stage developmental transition from CD11b^{low}CD27^{low} to CD11b^{high}CD27low (ref. ¹²) (FIG. 1d and FIG. 5g).

To assess the role of IL-1R8 in NK cells, inventors took advantage of IL-1R8-deficient mice. Among CD45⁺ cells, the NK cell frequency and absolute numbers were significantly higher in peripheral blood of Il1r8^{-/-} compared with Il1r8^{+/+} mice, and slightly increased in liver and spleen. (FIG. 2a, b). In addition, the frequency of the CD11b^{high}CD27^{low} and KLRG1⁺ mature subset was significantly higher in Il1r8^{-/-} mice than Il1r8^{+/+} mice in bone marrow, spleen and blood, indicating a more mature phenotype of NK cells¹³ (FIG. 2c, d and FIG. 6a, b).

The enhanced NK cell maturation in Il1r8^{-/-} mice occurred already at 2 and 3 weeks of age, whereas the frequency of NK precursors was similar in Il1r8^{-/-} and Il1r8^{+/+} bone marrow, indicating that IL-1R8 regulated early events in NK cell differentiation, but did not affect the development of NK cell precursors¹² (FIG. 6c-e).

Inventors next investigated whether IL-1R8 affected NK cell function. The expression of the activating receptors NKG2D, DNAM-1 and Ly49H was significantly upregulated in peripheral blood Il1r8^{-/-} NK cells (FIG. 6f). Interferon-γ (IFNγ) and granzyme B production and FasL expression were more sustained in IL-1R8-deficient NK cells upon ex vivo stimulation in the presence of IL-18 (FIG. 2e-g and FIG. 6g). The frequency of IFNγ⁺ NK cells was higher in Il1r8^{-/-} total NK cells and in all NK cell subsets. Thus, IFNγ production was enhanced independently of the NK cell maturation state. Analysis of competitive bone marrow chimaeras revealed that IL-1R8 regulates NK cell differentiation in a cell-autonomous way (FIG. 6h-k). Along the same line, co-culture experiments of NK cells with lipopolysaccharide (LPS) or CpG-primed dendritic cells showed that Il1r8^{-/-} NK cells produced higher IFNγ levels irrespective of the dendritic cell genotype (FIG. 6l).

IL-18 is a member of the IL-1 family, which plays an important role in NK cell differentiation and function^{1,14}. Enhanced NK cell maturation and effector function in Il1r8^{-/-} mice was abolished by IL-18 blockade or genetic deficiency but unaffected by IL-1R1-deficiency (FIG. 2h, i and FIG. 7a, b). Co-housing and antibiotic treatment had no impact, thus excluding a role of microbiota⁵ in the phenotype of Il1r8^{-/-} mice (FIG. 7c, d).

The results reported above suggested that IL-1R8 regulated the IL-18 signalling pathway in NK cells and, indeed, an increased phospho-IRAK4/IRAK4 ratio was induced by IL-18 in Il1r8^{-/-} NK cells compared with wild-type NK cells, indicating unleashed early signalling downstream of MyD88 and myddosome formation (FIG. 2j), consistent with the proposed molecular mode of action of IL-1R8 (refs 1, 9, 16). Indeed, by stimulated emission depletion (STED) microscopy, inventors observed clustering of IL-1R8 and IL-18Rα (FIG. 7e), in line with previous studies⁹. IL-1R8-deficiency also led to enhanced IL-18-dependent phosphorylation of S6 and JNK in NK cells, suggesting that IL-1R8 inhibited IL-18-dependent activation of the mTOR and JNK pathways (FIG. 2j), which control NK cell metabolism, differentiation and activation^{17,18}.

To obtain a deeper insight into the impact of IL-1R8 deficiency on NK cell function and on the response to IL-18, RNA sequencing (RNA-seq) analysis was conducted. IL-1R8 deficiency had a profound impact on the resting transcriptional profile of NK cells and on top on responsiveness to IL-18 (FIG. 2k, FIG. 8a and data deposited in the NCBI Gene Expression Omnibus under accession number GSE105043). The profile of IL-1R8-deficient cells includes activation pathways (for example, MAPK), adhesion molecules involved in cell-to-cell interactions and cytotoxicity (ICAM-1), and increased production of selected chemokines (CCL4). The last of these may represent an NK-cell-based amplification loop of leukocyte recruitment, including NK cells themselves.

To investigate the role of IL-1R8 in human NK cells (FIG. 1a, b), inventors first retrospectively analysed its expression in relation to responsiveness to a combination of IL-18 and IL-12 in normal donors. Inventors observed an inverse correlation between IL-1R8 levels and IFNγ production by peripheral blood NK cells (r²=0.7969, P=0.0012) (FIG. 2l). In addition, IL-1R8 partial silencing in peripheral blood NK cells with small interfering RNA (siRNA) was associated with a significant increase in IFNγ production (FIG. 2m) and upregulation of CD69 expression (not shown). These results suggest that in human NK cells, as in mouse counterparts, IL-1R8 serves as a negative regulator of activation and that its inactivation unleashes human NK-cell effector function.

To assess the actual relevance of IL-1R8-mediated regulation of NK cells, anticancer and antiviral resistance were examined. The liver is characterized by a high frequency of NK cells¹⁹ Therefore inventors focused on liver carcinogenesis. In a model of diethylnitrosamine-induced hepatocellular carcinoma, IL-1R8-deficient male and female mice²⁰ were protected against the development of lesions, in terms of macroscopic number, size (FIG. 3a and FIG. 9a, b) and histology (data not shown). The percentage and absolute number of NK cells, and the percentage of IFNγ+NK cells, were higher in Il1r8^{-/-} hepatocellular carcinoma-bearing mice (FIG. 3b, c and FIG. 9c). Finally, increased levels of cytokines involved in anti-tumour immunity (for example, IFNγ) and a reduction of pro-inflammatory cytokines associated with tumour promotion (IL-6, tumour necrosis factor-α, IL-1β, CCL2, CXCL1) were observed (Table 1). Most importantly, the depletion of NK cells abolished the protection against liver carcinogenesis observed in Il1r8^{-/-} mice (FIG. 3d and FIG. 9d).

Evidence suggests that NK cells can inhibit haematogenous cancer metastasis⁵. In a model of sarcoma (MN/MCA1) spontaneous lung metastasis, Il1r8^{-/-} mice showed a reduced number of haematogenous metastases, whereas primary tumour growth was unaffected (FIG. 3e and FIG. 9e, f). The frequency of total and mature CD27^{low} NK cells was higher in Il1r8^{-/-} lungs (FIG. 3f).

Assessment of lung metastasis at the time of euthanasia and in vivo imaging analysis (FIG. 3g and FIG. 9e) showed that the protection was completely abolished in NK-cell-depleted Il1r8^{-/-} mice. In addition, IL-18 or IFNγ neutralization abolished or markedly reduced the protection against metastasis observed in Il1r8^{-/-} mice (FIG. 9g). In contrast, depletion of CD4⁺/CD8⁺ cells or IL-17A, or deficiency of IL-1R1 (involved in T helper 17 cell development), did not affect the phenotype (FIG. 9h, i).

Liver metastasis is a major problem in the progression of colorectal cancer. Inventors therefore assessed the potential of Il1r8^{-/-} NK cells to protect against liver metastasis using the MC38 colon carcinoma line²¹. As shown in FIG. 3h, Il1r8^{-/-} mice were protected against MC38 colon carcinoma liver metastasis. In addition, IL-18 genetic deficiency abrogated the protection against liver metastasis observed in Il1r8^{-/-} mice (FIG. 9j), thus indicating that the IL-1R8-dependent control of MC38-derived liver metastasis occurs through the IL-18/IL-18R axis. To assess the primary role of Il1r8^{-/-} NK cells in the cancer protection, adoptive transfer was used (FIG. 9k-m). Adoptive transfer of Il1r8^{+/+} NK cells had no effect on lung and liver metastasis. In contrast, adoptive transfer of Il1r8^{-/-} NK cells significantly and markedly reduced the number and volume of lung and liver metastases (FIG. 3i, j and FIG. 9n). Given the natural history and clinical challenges of colorectal cancer, this observation has potential translational implications. Thus, IL-1R8 genetic inactivation unleashes NK-cell-mediated resistance to carcinogenesis in the liver and amplifies the anti-metastatic potential of these cells in liver and lung in a NK-cell-autonomous manner.

Finally, inventors investigated whether IL-1R8 affects NK cell antiviral activity, focusing on murine cytomegalovirus (MCMV) infection²². As shown in FIG. 4a, liver viral titres were lower in Il1r8^{-/-} than Il1r8^{+/+} mice, indicating that IL-1R8-deficiency was associated with a more efficient control of MCMV infection. The frequency of IFNγ⁺ NK cells and degranulation (that is, the frequency of CD107a⁺ NK cells) were significantly higher in the spleen and liver of Il1r8^{-/-} mice on day 1.5 after infection (FIG. 4b). On day 4.5 after infection, IFNγ⁺ and CD107a⁺ NK cells were strongly reduced, in both spleen and liver, as a consequence of better control of viral spread (FIG. 4b). Consistent with a more efficient control of the infection, reduced levels of pro-inflammatory cytokines were observed in Il1r8^{-/-} mice (FIG. 10a). NK-cell adoptive transfer experiments were performed in MCMV-infected newborn mice that still did not have mature NK cells¹². As shown in FIG. 4c, the adoptive transfer of Il1r8^{-/-} NK cells conferred higher protection than Il1r8^{+/+} NK cells, with for instance four out of nine mice having no detectable virus titre in the brain.

NK cells belong to the complex, diverse realm of innate lymphoid cells (ILCs)²³. Human and mouse non-NK ILCs express IL-1R8 mRNA and protein (ref. 24). Preliminary experiments were conducted to assess the role of IL-1R8 in ILC function. In the MCMV infection model, Il1r8^{-/-} ILC1 showed increased IFNγ production, but represented a minor population compared with NK cells and one-thirtieth that of Il1r8^{-/-} IFNγ-producing cells (FIG. 4d); they are therefore unlikely to play a significant role in the phenotype. These results provide initial evidence that IL-1R8 has a regulatory function in ILCs. Further studies are required to assess its actual relevance in ILC diverse populations. Collectively, these results indicate that IL-1R8-deficient mice were protected against MCMV infection and that protection was dependent on increased NK cell activation.

IL-1R8 deficiency was associated with exacerbated inflammatory and immune reactions under a variety of conditions^{1,10}. NK cells engage in bidirectional interactions with macrophages, dendritic cells and other lymphocytes^{3,4,25,26}. Therefore the role of NK cells in inflammatory and autoimmune conditions associated with IL-1R8 deficiency^{1,10} will need to be examined. IL-1R8-deficient mice show increased susceptibility to colitis and colitis-associated azoxymethane carcinogenesis^{27,28}. The divergent impact on carcinogenesis of IL-1R8 deficiency in the intestine and liver is likely to reflect fundamental, tissue-dictated differences of immune mechanisms involved in carcinogenesis in these different anatomical sites. In particular, high numbers of NK cells are present in the liver¹⁹ and this physiological characteristic of this organ is likely to underlie this apparent divergence.

NK cells are generally not credited with playing a major role in the control of solid tumours⁶. Conversely there is evidence for a role of NK cells in the control of haematogenous lung metastasis^{5,29.} The results presented here show that unleashing NK cells by genetic inactivation of IL-1R8 resulted in inhibition of liver carcinogenesis and protection against liver and lung metastasis. IL-1R8-deficient mice show exacerbated TLR and IL-1-driven inflammation¹⁰, and inflammation promotes liver carcinogenesis 30. Therefore, our results are probably an underestimate of the potential of removal of the NK cell checkpoint IL-1R8 against liver primary and metastatic tumours. Thus, NK cells have the potential to restrain solid cancer and metastasis, provided critical, validated checkpoints such as IL-1R8 are removed and the tissue immunological landscape is taken into account.

### EXAMPLE 2

Materials and Methods

In Vitro NK Cell Functional Activation

Il1r8+/+ and Il1r8−/− splenic NK cells were enriched using a negative magnetic separation (NK cell isolation kit II, Miltenyi) (as described in example 1) and cultured for 8 days in RPMI 10% FBS with IL-2 (Peprotech, 20 ng/ml) plus IL-15 (Peprotech, 10 ng/ml) (Huang B Y et al, PloS ONE (2015). MC38 cells (as described in example 1) were pre-treated (24 hours) with IFNγ, in order to mimic the tumor microenvironment and induce the expression of PD-L1, as previously shown (Juneja V R et al, J. Exp. Med. (2017). NK cells were pre-incubated for 30 minutes (37° C.) with anti-PD1 blocking antibody or the relative isotype control (both BioxCell, 1 μg/ml). MC38 cells were washed and co-cultured with NK cells (1:2 ratio) for 3 hours. IFNγ and GranzymeB intracellular expression in NK cells was measured by flow cytometry.

Results

Effect In Vitro of the Combination of IL-1R8-Deficiency and PD-1 Blockade

Inventors herein show that the blockade of PD-1 drives an increased NK cell activation in IL-1R8-deficient NK cells compared to wild-type NK cells, when exposed to a tumoral target expressing the ligand (PD-L1), demonstrating that the combination of IL-1R8 and PD-1 blockade enforces NK cell effector functions (FIG. 12).

### EXAMPLE 3

Materials and Methods

IL-1R8 Expression in Human T Cells

Human peripheral mononuclear cells (PBMCs) were isolated from peripheral blood of healthy donors through a Ficoll density gradient centrifugation (GE Healthcare Biosciences), upon approval by Humanitas Research Hospital Ethical Committee. IL-1R8 expression was measured by flow cytometry in T cell subsets according to the expression of CD3, CD4, CD8, CCR7, CD45RO, CD127, CD25 (Gattinoni L. et al. Nature Medicine (2011).

Proliferation Assay

Il1r8+/+ and Il1r8−/− murine splenic T were enriched using a negative magnetic separation (Pan T cell isolation kit II, Miltenyi) and pre-incubated for 10 minutes (37° C.) with Vybrant® CFDA SE dye (Invitrogen, 1 μM). T cells were washed and cultured for 2 days in IMDM 10% FBS 0.1% BME (Gibco) with Dynabeads Mouse T-Activator CD3/CD28 (Gibco, 1 bead×cell) plus IL-2 (Proleukin, 20 ng/ml), IL-12 (Peprotech, 20 ng/ml), IL-18 (MBL, 20 ng/ml) alone or in combination (Hu B. et al. Cell Rep (2017); Freeman B. et al. PNAS (2012)). CFDA SE and CD44 expression in CD8 T cells was measured by flow cytometry.

T Cell Activation In Vitro

Il1r8+/+ and Il1r8−/− murine splenic CD8+ T cells were enriched using a negative magnetic separation (CD8a+ isolation kit, mouse, Miltenyi) and cultured for 2 days in IMDM 10% FBS 0.1% BME (Gibco) with Dynabeads Mouse T-Activator CD3/CD28 (Gibco, 1 bead×cell) plus IL-2 (Proleukin, 20 ng/ml), IL-12 (Peprotech, 20 ng/ml) alone or in combination. T cells were treated (overnight) with IL-18 (MBL, 20 ng/ml) and stimulated for 3 h with Cell Stimulation Cocktail (eBioscience) plus Golgi Plug (BD Biosciences) as specified (Hu B. et al. Cell Rep (2017); Freeman B. et al. PNAS (2012)). IFNγ and GranzymeB intracellular expression in CD8 T cells was measured by flow cytometry.

Results

Inventors hypothesized that CD8+T lymphocytes expressed IL-1R8 and that it played a negative regulatory activity in this cell type. Inventors first checked IL-1R8 expression in human T cells from healthy donors by flow cytometry. Here inventors show that human CD8+ T cells display a higher level of IL-1R8 compared to CD4+ T cells. Moreover, IL-1R8 expression is increased in effector/memory T cell subsets compared with naïve T cells, demonstrating that IL-1R8 expression is associated with the acquisition of the effector potential (FIG. 13). To elucidate the role of IL-1R8 in cytotoxic CD8+ T cells, inventors assessed CD8+ T cell proliferation, maturation and activation in vitro upon TCR stimulation, in combination with the cytokines IL-2, IL-12 and IL-18, which are involved in CD8+ T cell activation. In FIG. 14A inventors show that Il1r8−/− CD8+ T cells exhibit a higher proliferation rate compared to CD8+ T cells from wt mice. In line with this observation, the maturation marker CD44 is upregulated in Il1r8−/− CD8+ T cells compared to wt CD8+ T cell (FIG. 14B), suggesting that IL-1R8 deficiency promotes CD8+ T cell expansion and the transition from naïve to effector T cells. Finally, inventors show that IFNγ and Granzyme B production is enhanced in Il1r8−/− CD8+ T cells and that IL-1R8-deficiency increases the response to IL-18 stimulation (FIG. 15A-D). These results indicate that IL-1R8 genetic silencing leads to increased CD8+ T cell proliferation, maturation and activation.

### REFERENCES

1. Garlanda, C., Dinarello, C. A. & Mantovani, A. The interleukin-1 family: back to the future. Immunity 39, 1003-1018 (2013).
2. Di Santo, J. P. Natural killer cell developmental pathways: a question of balance. Annu. Rev. Immunol. 24, 257-286 (2006).
3. Vivier, E. et al. Innate or adaptive immunity? The example of natural killer cells.

Science 331, 44-49 (2011).
4. Bellora, F. et al. Human NK cells and NK receptors. Immunol. Lett. 161, 168-173 (2014).
5. Guillerey, C., Huntington, N. D. & Smyth, M. J. Targeting natural killer cells in cancer immunotherapy. Nat. Immunol. 17, 1025-1036 (2016).
6. Stojanovic, A. & Cerwenka, A. Natural killer cells and solid tumors. J. Innate Immun. 3, 355-364 (2011).
7. Gismondi, A., Stabile, H., Nisti, P. & Santoni, A. Effector functions of natural killer cell subsets in the control of hematological malignancies. Front. Immunol. 6, 567 (2015).
8. Gulen, M. F. et al. The receptor SIGIRR suppresses Th17 cell proliferation via inhibition of the interleukin-1 receptor pathway and mTOR kinase activation. Immunity 32, 54-66 (2010).
9. Nold-Petry, C. A. et al. IL-37 requires the receptors IL-18R□ and IL-1R8 (SIGIRR) to carry out its multifaceted anti-inflammatory program upon innate signal transduction. Nat. Immunol. 16, 354-365 (2015).
10. Garlanda, C., Riva, F., Bonavita, E. & Mantovani, A. Negative regulatory receptors of the IL-1 family. Semin. Immunol. 25, 408-415 (2013).
11. Cooper, M. A. et al. Human natural killer cells: a unique innate immunoregulatory role for the CD56bright subset. Blood 97, 3146-3151 (2001).
12. Chiossone, L. et al. Maturation of mouse NK cells is a 4-stage developmental program. Blood 113, 5488-5496 (2009).
13. Kim, S. et al. In vivo developmental stages in murine natural killer cell maturation. Nat. Immunol. 3, 523-528 (2002).
14. Takeda, K. et al. Defective NK cell activity and Th1 response in IL-18-deficient mice. Immunity 8, 383-390 (1998).
15. Ganal, S. C. et al. Priming of natural killer cells by nonmucosal mononuclear phagocytes requires instructive signals from commensal microbiota. Immunity 37, 171-186 (2012).
16. Gong, J. et al. Inhibition of Toll-like receptors TLR4 and 7 signaling pathways by SIGIRR: a computational approach. J. Struct. Biol. 169, 323-330 (2010).
17. Margais, A. et al. The metabolic checkpoint kinase mTOR is essential for IL-15 signaling during the development and activation of NK cells. Nat. Immunol. 15, 749-757 (2014).
18. Li, C. et al. JNK MAP kinase activation is required for MTOC and granule polarization in NKG2D-mediated NK cell cytotoxicity. Proc. Natl Acad. Sci. USA 105, 3017-3022 (2008).
19. Peng, H. & Tian, Z. Re-examining the origin and function of liver-resident NK cells. Trends Immunol. 36, 293-299 (2015).
20. Naugler, W. E. et al. Gender disparity in liver cancer due to sex differences in MyD88-dependent IL-6 production. Science 317, 121-124 (2007).
21. Dupaul-Chicoine, J. et al. The Nlrp3 inflammasome suppresses colorectal cancer metastatic growth in the liver by promoting natural killer cell tumoricidal activity. Immunity 43, 751-763 (2015).
22. Lisnić, B., Lisnić, V. J. & Jonjić, S. NK cell interplay with cytomegaloviruses. Curr. Opin. Virol. 15, 9-18 (2015).
23. Eberl, G., Colonna, M., Di Santo, J. P. & McKenzie, A. N. Innate lymphoid cells: a new paradigm in immunology. Science 348, aaa6566 (2015).
24. Shih, H. Y. et al. Developmental acquisition of regulomes underlies innate lymphoid cell functionality. Cell 165, 1120-1133 (2016).
25. Bellora, F. et al. M-CSF induces the expression of a membrane-bound form of IL-18 in a subset of human monocytes differentiating in vitro toward macrophages. Eur. J. Immunol. 42, 1618-1626 (2012).
26. Martin-Fontecha, A. et al. Induced recruitment of NK cells to lymph nodes provides IFN-gamma for T(H)1 priming. Nat. Immunol. 5, 1260-1265 (2004).
27. Garlanda, C. et al. Increased susceptibility to colitis-associated cancer of mice lacking TIR8, an inhibitory member of the interleukin-1 receptor family. Cancer Res. 67, 6017-6021 (2007).
28. Xiao, H. et al. The Toll-interleukin-1 receptor member SIGIRR regulates colonic epithelial homeostasis, inflammation, and tumorigenesis. Immunity 26, 461-475 (2007).
29. Morvan, M. G. & Lanier, L. L. NK cells and cancer: you can teach innate cells new tricks. Nat. Rev. Cancer 16, 7-19 (2016).
30. He, G. & Karin, M. NF-κB and STAT3—key players in liver inflammation and cancer. Cell Res. 21, 159-168 (2011).
31. Garlanda, C. et al. Intestinal inflammation in mice deficient in Tir8, an inhibitory member of the IL-1 receptor family. Proc. Natl Acad. Sci. USA 101, 3522-3526 (2004).
32. Bushnell, B. Bbmap: a fast, accurate, splice-aware aligner (Ernest Orlando Lawrence Berkeley National Laboratory, 2014).
33. Martin, M. Cutadapt removes adapter sequences from high-throughput sequencing reads. EMBnet.journal 17, (2011).
34. Dobin, A. et al. STAR: ultrafast universal RNA-seq aligner. Bioinformatics 29, 15-21 (2013).
35. Gentleman, R. C. et al. Bioconductor: open software development for computational biology and bioinformatics. Genome Biol. 5, R80 (2004).
36. Robinson, M. D., McCarthy, D. J. & Smyth, G. K. edgeR: a Bioconductor package for differential expression analysis of digital gene expression data. Bioinformatics 26, 139-140 (2010).
37. Majewski, I. J. et al. Opposing roles of polycomb repressive complexes in hematopoietic stem and progenitor cells. Blood 116, 731-739 (2010).
38. Ritchie, M. E. et al. limma powers differential expression analyses for RNA-sequencing and microarray studies. Nucleic Acids Res. 43, e47 (2015).
39. Mingozzi, F. et al. Prolonged contact with dendritic cells turns lymph node-resident NK cells into anti-tumor effectors. EMBO Mol. Med. 8, 1039-1051 (2016).
40. Giavazzi, R., Alessandri, G., Spreafico, F., Garattini, S. & Mantovani, A. Metastasizing capacity of tumour cells from spontaneous metastases of transplanted murine tumours. Br. J. Cancer 42, 462-472 (1980).
41. Wagner, M., Jonjic, S., Koszinowski, U. H. & Messerle, M. Systematic excision of vector sequences from the BAC-cloned herpesvirus genome during virus reconstitution. J. Virol. 73, 7056-7060 (1999).
42. Jonji\tilde{c}, S., Pavić, I., Lucin, P., Rukavina, D. & Koszinowski, U. H. Efficacious control of cytomegalovirus infection after long-term depletion of CD8+T lymphocytes. J. Virol. 64, 5457-5464 (1990).
43. Reddehase, M. J. et al. Interstitial murine cytomegalovirus pneumonia after irradiation: characterization of cells that limit viral replication during established infection of the lungs. J. Virol. 55, 264-273 (1985).

1. An isolated human cell, being a natural killer (NK) cell or T cell, wherein said cell is stably or transiently deficient in the expression and/or activity of IL-1R8.

2. The cell according to claim 1, wherein said T cell is a CD8+ T cell.

3. The cell according to claim 1, wherein said cell produces greater amounts of at least one effector molecule involved in anti-tumour immunity than cells that do express IL-1R8.

4. The cell according to claim 3, wherein said molecule is interferon-gamma (IFN-γ) and/or granzyme B and/or FasL.

5. The cell according to claim 1, being further deficient in the expression and/or activity of at least one checkpoint for NK cell maturation and/or effector function.

6. The cell according to claim 5 wherein said at least one checkpoint for NK cell maturation and/or effector function is selected from the group consisting of: CIS, KIRs, PD-1, CTLA-4, TIM-3, NKG2A, CD96, and TIGIT.

7. A population of cells comprising the NK cells and/or T cells as defined in claim 1.

8. A composition comprising the cells as defined in claim 1, said composition optionally further comprising at least one physiologically acceptable carrier.

9. The cell according to claim 1 for use as a medicament, optionally for use in the treatment and/or prevention of tumour and/or metastasis, or of microbial or viral infection.

10. The cell according to claim 9 being used in Adoptive cell transfer (ACT), cell therapy treatment, mismatched bone marrow transplantation, mismatched NK cell infusion or cytokine-induced killer (CIK) cell infusion.

11. (canceled)

12. A suppressor or inhibitor of IL-1R8 expression and/or activity for use in the treatment and/or prevention of tumour and/or metastasis, or of microbial or viral infection.

13. The suppressor or inhibitor according to claim 12, wherein the suppressor or inhibitor is at least one molecule selected from the group consisting of:
a) an antibody or a fragment thereof;
b) a polypeptide;
c) a small molecule;
d) a polynucleotide coding for said antibody or polypeptide or a functional derivative thereof;
e) a polynucleotide, such as antisense construct, antisense oligonucleotide, RNA interference construct or siRNA,
f) a vector comprising or expressing the polynucleotide as defined in d) or e);
g) a CRISPR/Cas9 component, e.g. a sgRNA;
h) a host cell genetically engineered expressing said polypeptide or antibody or comprising the polynucleotide as defined in d) or e) or at least one component of g),
optionally said polynucleotide being an RNA inhibitor, optionally selected from the group consisting of: siRNA, miRNA, shRNA, stRNA, snRNA, and antisense nucleic acid, more optionally the polynucleotide is at least one siRNA selected from the group consisting of: AGU UUC GCG AGC CGA GAU CUU (SEQ ID NO: 1); UAC CAG AGC AGC ACG UUG AUU (SEQ ID NO:2); UGA CCC AGG AGU ACU CGU GUU (SEQ ID NO:3); CUU CCC GUC GUU UAU CUC CUU (SEQ ID NO:4) (all 5′ to 3′), or a functional derivative thereof.

14. The suppressor according to claim 11, being used in NK and/or T cells.

15. The suppressor or inhibitor according claim 12, being used in Adoptive cell transfer (ACT), cell therapy treatment, mismatched bone marrow transplantation, mismatched NK cell infusion or cytokine-induced killer (CIK) cell infusion.

16. A pharmaceutical composition comprising the suppressor or inhibitor as defined in claim 12 and at least one pharmaceutically acceptable carrier, and optionally further comprising a therapeutic agent.

17. The cell according to claim 9, wherein:
a) the tumour is a solid tumor or an hematological tumor, optionally selected from the group consisting of: Colon/Rectum Cancer, Adrenal Cancer, Anal Cancer, Bile Duct Cancer, Bladder Cancer, Bone Cancer, Brain/CNS Tumors In Adults, Brain/CNS Tumors In Children, Breast Cancer, Breast Cancer In Men, Cancer of Unknown Primary, Castleman Disease, Cervical Cancer, Endometrial Cancer, Esophagus Cancer, Ewing Family Of Tumors, Eye Cancer, Gallbladder Cancer, Gastrointestinal Carcinoid Tumors, Gastrointestinal Stromal Tumor (GIST), Gestational Trophoblastic Disease, Hodgkin Disease, Kaposi Sarcoma, Kidney Cancer, Laryngeal and Hypopharyngeal Cancer, Leukemia, Acute Lymphocytic (ALL), Acute Myeloid (AML, including myeloid sarcoma and leukemia cutis), Chronic Lymphocytic (CLL), Chronic Myeloid (CML) Leukemia, Chronic Myelomonocytic (CMML), Leukemia in Children, Liver Cancer, Lung Cancer, Lung Cancer with Non-Small Cell, Lung Cancer with Small Cell, Lung Carcinoid Tumor, Lymphoma, Lymphoma of the Skin, Malignant Mesothelioma, Multiple Myeloma, Myelodysplastic Syndrome, Nasal Cavity and Paranasal Sinus Cancer, Nasopharyngeal Cancer, Neuroblastoma, Non-Hodgkin Lymphoma, Non-Hodgkin Lymphoma In Children, Oral Cavity and Oropharyngeal Cancer, Osteosarcoma, Ovarian Cancer, Pancreatic Cancer, Penile Cancer, Pituitary Tumors, Prostate Cancer, Retinoblastoma, Rhabdomyosarcoma, Salivary Gland Cancer, Sarcoma—Adult Soft Tissue Cancer, Skin Cancer, Skin Cancer—Basal and Squamous Cell, Skin Cancer—Melanoma, Skin Cancer—Merkel Cell, Small Intestine Cancer, Stomach Cancer, Testicular Cancer, Thymus Cancer, Thyroid Cancer, Uterine Sarcoma, uveal melanoma, Vaginal Cancer, Vulvar Cancer, Waldenstrom Macroglobulinemia, Wilms Tumor, more optionally the tumour is a solid tumor, optionally colorectal cancer, and the metastasis are lung or liver metastasis or
b) the infection is caused by one of the following viruses or bacteria: herpesviruses, optionally cytomegalovirus, Human Immunodeficiency Virus (HIV), Hepatitis C Virus (HCV), Hepatitis B Virus (HBV), West Nile virus (WNV), Salmonella, Shigella, Legionella, Mycobacterium.

18. A method to obtain the cell according to claim 1, comprising the step of stably or transiently inhibiting or suppressing the expression and/or function of IL-1R8 in an NK or T cell or cell population comprising NK and/or T cells and optionally further expanding in vitro the silenced population.

19. The method according to claim 18 wherein said T cell is a CD8+ T cell.

20. The method according to claim 18, wherein said NK or T cell or cell population is optionally previously purified from isolated peripheral blood mononuclear cell (PBMCs) and optionally expanded in vitro, optionally using rhIL-2.

21. The method according to claim 18 further comprising the inhibition or suppression of the expression and/or function of at least one further checkpoint for NK cell maturation and/or effector function.

22. The method according to claim 21 wherein said at least one checkpoint for NK cell maturation and/or effector function is selected from the group consisting of: CIS, KIRs, PD-1, CTLA-4, TIM-3, NKG2A, CD96, and TIGIT.
